(19) United States
(12) Patent Application Publication
(10) Pub. No.: US 2017/0002097 A1
(43) Pub. Date: Jan. 5, 2017

(54) MULTI-SPECIFIC BINDING PROTEINS

(71) Applicant: Boehringer Ingelheim International GmbH, Ingelheim am Rhein (DE)

(72) Inventors: Rajkumar GANESAN, Ridgefield, CT (US); Sanjaya SINGH, Sandy Hook, CT (US); Abdulsalam SHAABAN, Danbury, CT (US)

(21) Appl. No.: 15/196,694

(22) Filed: Jun. 29, 2016

Related U.S. Application Data
(60) Provisional application No. 62/186,423, filed on Jun. 30, 2015.

(51) Int. Cl. C07K 16/46; C07K 16/24

(52) U.S. Cl. CPC C07K 16/468; C07K 16/241; C07K 16/244; C07K 2317/31; C07K 2317/51; C07K 2317/515; C07K 2317/622; C07K 2317/94

(57) ABSTRACT

This invention generally relates to multi-specific binding proteins. The invention also relates to methods of making such proteins and to methods of using such proteins. Pharmaceutical compositions and kits comprising such proteins are also disclosed.

### TECHNICAL FIELD OF THE INVENTION

This invention generally relates to multi-specific binding proteins. The invention also relates to methods of making such proteins and to methods of using such proteins. Pharmaceutical compositions and kits comprising such proteins are also disclosed.

### BACKGROUND OF THE INVENTION

Monoclonal antibodies as a monotherapy have been used with considerable success for the treatment of various diseases, including cancer and immunological diseases. Their ability to bind specifically to their target has led to medical advances. However, in some therapies, the modulation of more than one target may be beneficial and biological molecules that bind to more than one target protein or to different epitopes on a target protein may offer additional benefits when compared to monoclonal antibodies.

A number of designs for biological structures that bind to more than one target have been proposed, but the development of multi-specific biological molecules can be challenging. The most common method of generating bispecific molecules is by genetic fusion of antibody fragments by polypeptide linkers. Due to the symmetrical nature of the IgGs, antibody domain fusion bispecifics are bivalent in nature. However, in certain instances, bivalency leads to undesired agonistic activity. Multimerization domains, such as leucine zippers, have been used to force two binding specificities into a single molecule. While linkers have advantages for the engineering of bispecific molecules, they may also cause problems in therapeutic settings. Indeed, these foreign peptides might elicit an immune response against the linker itself or the junction between the protein and the linker. Such structures may also have reduced stability in-vivo and/or be difficult to express, leading to lack of homogeneity or to the production of partial amino acid chains.

Other strategies have been designed to create heterodimers of two different heavy chains. However, these strategies are hampered by the formation of substantial amounts of undesired homodimers of each of the heavy chains and by the mis-pairing of the light chains. Additional difficulties with multi-specific structures are also a reduction in functionality, e.g. reducing affinity to the target.

Thus in summary, design and development of bispecific biological molecules pose a number of challenges, and there is a need for multi-specific binding proteins having adequate pharmacological properties and which can be manufactured effectively.

Accordingly, one aim of the present invention is to provide multi-specific binding proteins which have favorable biophysical and/or pharmacological properties.

A further aim of the present invention is to provide multi-specific binding proteins, which can be produced at high levels of homogeneity and/or integrity.

A further aim of the present invention is to provide multi-specific binding proteins, which can be produced effectively, for example in mammalian cells.

A further aim of the present invention is to provide multi-specific binding proteins that maintain the functionality of their binding moieties.

A further aim of the present invention is to provide multi-specific binding proteins, which allow flexibility in the selection of binding moities.

A further aim of the present invention is to provide multi-specific binding proteins, which avoid undesired immune responses.

A further aim of the present invention is to provide multi-specific binding proteins, which have favorable developability properties, such as stability.

Further aims of the present invention include combinations of any of the aims set forth above.

### SUMMARY OF THE INVENTION

The present invention addresses the above needs and provides proteins comprising at least two binding units that are specific to two different epitopes. In one aspect, a protein the present invention comprises a first heavy chain and a first light chain forming a first binding unit specific for a first epitope and a second heavy chain and a second light chain forming a second binding unit specific for a second epitope. In one aspect, the first heavy chain and said second heavy chain each comprises one or more amino acid changes which reduces the formation of homodimers of one of the heavy chains. In one aspect, such amino acid changes are a tyrosine (Y) at position 366 [T366Y, EU numbering (Edelman et al, Proc Natl Acad Sci USA. 1969 May; 63(1):78-85) of the first heavy chain and a threonine (T) at position 407 [Y407T, EU numbering] of the second heavy chain. In one aspect, such amino acid changes are a tryptophan (W) at position 366 [T366W] of the first heavy chain and a serine (S) at position 366 [T366S], an alanine (A) at position 368 [L368A] and a valine (V) at position 407 [Y407V] of the second heavy chain. In one aspect, the heavy chains are heavy chains derived from the heavy chain of an IgG₁ or IgG₄. In one aspect, the first heavy chain comprises a cysteine (C) at position 354 [S354C] in addition to the tryptophan (W) at position 366 [T366W] and the second heavy chain comprises a cysteine (C) at position 349 [Y349C] in addition the serine (S) at position 366 [T366S], the alanine (A) at position 368 [L368A] and the valine (V) at position 407 [Y407V]. In one aspect, the heavy chains are heavy chains derived from the heavy chain of an IgG₄. The inclusion of these amino acid changes in the two heavy chains facilitates heterodimerization of the two heavy chains and minimize the formation of homodimers. These amino acid changes also have low immunogenicity based on the in silico assessment (De Groot et al. Trends Immunol. 2007 November; 28(11):482. In one aspect, the first heavy chain or the second heavy chain in a protein of the present invention further comprises one or more amino acid changes which reduce the binding of the heavy chain to staphylococcal Protein A. In one aspect, such amino acid changes are an arginine at position 435 [H435R, EU numbering] and a phenylalanine at position 436 [Y436F, EU numbering] of one of the heavy chains. These two mutations are located in the CH3 domain and are incorporated in one of the heavy chains to reduce binding to Protein A. These two changes facilitate the removal of homodimers of heavy chains and other impurities during protein purification. In one aspect, in a protein of the present invention, the arginine at position 435 [H435R] and the phenylalanine at position 436 [Y436F] are comprised in the heavy chain, which also comprises a threonine (T) at position 407 [Y407T]. In one aspect, in a protein of the present invention, the arginine at position 435 [H435R] and the phenylalanine at position 436 [Y436F] are comprised in the heavy chain, which also comprises a serine (S) at position 366 [T366S], an alanine (A) at position 368 [L368A] and a valine (V) at position 407 [Y407V].

In a further aspect, the first and second heavy chains form a heterodimer through one or more di-sulfide bridges in a protein of the present invention.

In a further aspect, in a protein if the present invention, the heavy chain and the light chain in one of the binding units are covalently linked through a linker. In one aspect, the heavy chain and the light chain in the two binding units are respectively covalently linked through a linker. This averts mis-pairing of the light chains during expression and purification of proteins, and allows the use of a wide variety of light chains in a protein of the present invention without compromising the functionality and/or bind affinity of the binding units.

In one aspect, a linker used in a protein of the present invention comprises 26 to 42 amino acids, for example 30 to 40 amino acids. In a further aspect, a linker used in a protein of the present invention comprises 34 to 40 amino acids, for example 36 to 39 amino acids, for example 38 amino acids.

Accordingly, in one aspect, the present invention provides a protein comprising a first amino acid chain and a second amino acid chain, wherein the first chain comprises a first light chain covalently linked to a linker, which is itself covalently linked to a first heavy chain, and wherein the second chain comprises a second light chain covalently linked to a linker, which is itself covalently linked to a second heavy chain.

In one aspect, starting from its N-terminus, the first chain comprises a light chain variable region, a light chain constant region, a linker, a heavy chain variable region and a heavy chain constant region. In one aspect, starting from its N-terminus, the second chain comprises a light chain variable region, a light chain constant region, a linker, a heavy chain variable region and a heavy chain constant region. In one aspect, both the first and the second chains comprise starting from their N-terminus a light chain variable region, a light chain constant region, a linker, a heavy chain variable region and a heavy chain constant region.

The resulting proteins bears a full Fc, which is marginally larger than an IgG and has two independent binding sites, for example each for one target protein or for an epitope on a target protein. This format greatly reduces heterogeneity after expression and purification and maintains the functional properties of the binding moieties. This also enables the expression of homogenous proteins, which express well, e.g. in mammalian cells. The proteins of the present invention have an acceptable immunogenicity profile and have satisfactory stability in-vitro and in-vivo.

The present invention further discloses nucleic acid sequences and DNA molecules encoding the amino acid sequences of a protein of the present invention. The present invention further discloses vectors, for example expression vectors, comprising such nucleic acid sequences and DNA molecules, and cells comprising such vectors. The present invention further discloses methods of producing proteins of the present invention and method of using such proteins, for example therapeutic methods.

The proteins of the present invention are useful in methods of treating or preventing diseases or disorders, for example as described herein. The disease or disorder treated or prevented will depend on the specificity of the binding units, that is the target protein(s) recognized by the binding units in a protein of the present invention.

Accordingly, the present invention also provides a method for treating a disease or disorder comprising administering to a patient a protein of the present invention. The present invention also provides a protein of the present invention for use in medicine, for example for treating or preventing a disease or disorder in mammals, in particular humans.

Accordingly, in one embodiment, the present invention provides a protein comprising:
a) a first heavy chain and a first light chain forming a first binding unit specific for a first epitope, and
b) a second heavy chain and a second light chain forming a second binding unit specific for a second epitope,
   wherein the first heavy chain comprises a tyrosine (Y) at position 366 [T366Y], wherein the second heavy chain comprises a threonine (T) at position 407 [Y407T], and wherein the first or the second heavy chain further comprises an arginine at position 435 [H435R] and a phenylalanine at position 436 [Y436F].

In one embodiment, the present invention provides a protein comprising:
a) a first heavy chain and a first light chain forming a first binding unit specific for a first epitope, and
b) a second heavy chain and a second light chain forming a second binding unit specific for a second epitope,
   wherein said first heavy chain comprises a tryptophan (W) at position 366 [T366W], wherein said second heavy chain comprises a serin (S) at position 366 [T366S], an alanine (A) at position 368 [L368A] and a valine (V) at position 407 [Y407V], and wherein said first or said second heavy chain further comprises an arginine at position 435 [H435R] and a phenylalanine at position 436 [Y436F].

In one embodiment, the heavy chains are derived from the heavy chain of an IgG₁ or IgG₄. In one embodiment, the heavy chains are derived from the heavy chain of an IgG₁. In one embodiment, the heavy chains are derived from the heavy chain of an IgG₄.

In one embodiment, the second heavy chain further comprises an arginine at position 435 [H435R] and a phenylalanine at position 436 [Y436F].

In one embodiment, the first and the second heavy chains further comprises YTE mutations (M252Y/S254T/T256E).

In one embodiment, the first heavy chain comprises the amino acid sequence of SEQ ID NO: 1, 4, 36 or 37. In one embodiment, the second heavy chain comprises the amino acid sequence of SEQ ID NO: 3, 5, 38 or 39. In one embodiment, the first heavy chain comprises the amino acid sequence of SEQ ID NO: 1 or 4 and the second heavy chain comprises the amino acid sequence of SEQ ID NO: 3 or 5.

In one embodiment, the first heavy chain comprises the amino acid sequence of SEQ ID NO: 36 and/or the second heavy chain comprises the amino acid sequence of SEQ ID NO: 38. In one embodiment, the first heavy chain comprises the amino acid sequence of SEQ ID NO: 37 and/or the second heavy chain comprises the amino acid sequence of SEQ ID NO: 39.

In one embodiment, the first and second heavy chains each further comprise a heavy chain variable region.

In one embodiment, the first or second light chain comprises the amino acid sequence of SEQ ID NO: 2 or 35. In one embodiment, the first light chain and the second light chain comprise the amino acid sequence of SEQ ID NO: 2. In one embodiment, the first light chain and the second light chain comprise the amino acid sequence of SEQ ID NO: 2. In one embodiment, the first and second light chains each further comprise a light chain variable region.

In one embodiment, a protein of the present invention comprises a first heavy chain comprising the amino acid sequence of SEQ ID NO: 1, a first light chain comprising the amino acid sequence of SEQ ID NO: 2, a second heavy chain comprising the amino acid sequence of SEQ ID NO: 3 and a second light chain comprising the amino acid sequence of SEQ ID NO: 2. In one embodiment, the first and second heavy chains each further comprise a heavy chain variable region and the first and second light chains each further comprise a light chain variable region.

In one embodiment, a protein of the present invention comprises a first heavy chain comprising the amino acid sequence of SEQ ID NO: 4, a first light chain comprising the amino acid sequence of SEQ ID NO: 2, a second heavy chain comprising the amino acid sequence of SEQ ID NO: 5 and a second light chain comprising the amino acid sequence of SEQ ID NO: 2. In one embodiment, the first and second heavy chains each further comprise a heavy chain variable region and the first and second light chains each further comprise a light chain variable region.

In one embodiment, a protein of the present invention comprises a first heavy chain comprising the amino acid sequence of SEQ ID NO: 36, a first light chain comprising the amino acid sequence of SEQ ID NO: 2, a second heavy chain comprising the amino acid sequence of SEQ ID NO: 38 and a second light chain comprising the amino acid sequence of SEQ ID NO: 2. In one embodiment, the first and second heavy chains each further comprise a heavy chain variable region and the first and second light chains each further comprise a light chain variable region.

In one embodiment, a protein of the present invention comprises a first heavy chain comprising the amino acid sequence of SEQ ID NO: 37, a first light chain comprising the amino acid sequence of SEQ ID NO: 2, a second heavy chain comprising the amino acid sequence of SEQ ID NO: 39 and a second light chain comprising the amino acid sequence of SEQ ID NO: 2. In one embodiment, the first and second heavy chains each further comprise a heavy chain variable region and the first and second light chains each further comprise a light chain variable region.

In one embodiment, the first and/or the second light chain comprises the amino acid sequence of SEQ ID NO:35 instead of the amino acid sequence of SEQ ID NO:2.

In one embodiment, the first heavy chain and the first light chain are covalently linked through a first linker. In one embodiment, the second heavy chain and the second light chain are covalently linked through a second linker. In one embodiment, the first heavy chain and the first light chain are covalently linked through a first linker and the second heavy chain and the second light chain are covalently linked through a second linker.

In one embodiment, the first and/or said second linker comprises 26 to 42 amino acids. In one embodiment, the first and/or said second linker comprises 30 to 40 amino acids. In one embodiment, the first and/or said second linker comprises 34 to 40 amino acids. In one embodiment, the first and/or said second linker comprises 36 to 39 amino acids. In one embodiment, the first and/or said second linker comprises 38 amino acids.

In one embodiment, the first and/or said second linker comprises glycine and serine amino acids. In one embodiment, the first and/or said second linker comprises the amino acid sequence of any one of SEQ ID NO:6 to SEQ ID NO:14 or SEQ ID NO:40.

In one embodiment, the first and said second linker have the same length. In one embodiment, the first linker and said second linker are identical. In one embodiment, the first and said second linker comprises the amino acid sequence of any one of SEQ ID NO:6 to SEQ ID NO:14 or SEQ ID NO:40.

In one embodiment, the first linker is covalently linked to the N-terminus of the first heavy chain and to the C-terminus of the first light chain and the second linker is covalently linked to the N-terminus of the second heavy chain and to the C-terminus of the second light chain.

In one embodiment, the first and the second epitopes are on the same target protein. In one embodiment, the first and the second epitopes are on different target proteins.

In one embodiment a protein of the present invention further comprises a third binding unit specific to a third epitope. In one embodiment, the third binding unit is covalently linked to the C-terminus of the first or second heavy chain. In one embodiment, the third binding unit is covalently linked to the N-terminus of the first or second light chain.

In one embodiment, the protein of the present invention further comprises a fourth binding unit specific to a fourth epitope. In one embodiment, the fourth binding unit is covalently linked to the C-terminus of the first or second heavy chain. In one embodiment, the fourth binding unit is covalently linked to the N-terminus of the first or second light chain. In one embodiment, the third and/or fourth binding unit is a scFv.

In one embodiment, the present invention further provides a pharmaceutical composition comprising a protein as described above and a pharmaceutically acceptable carrier.

In one embodiment, the present invention further provides an isolated polynucleotide comprising a sequence encoding a light chain or a heavy chain as described above.

In one embodiment, the present invention further provides an expression vector comprising a polynucleotide as described above.

In one embodiment, the present invention further provides a host cell comprising one or more isolated polynucleotide(s) as described above or one or more expression vector(s) as described above.

In one embodiment, the present invention further provides a method for producing protein comprising obtaining a host cell as described above and cultivating the host cell. In one embodiment, the method further comprises recovering and purifying the protein.

### BRIEF DESCRIPTION OF THE FIGURES

FIG. 1: Schematic diagram of a representative ZweiMab bi-specific antibody. The bi-specific antibody represented in FIG. 1 comprise amino acid changes to a tyrosine (Y) at position 366 [T366Y] of the first heavy chain and to a threonine (T) at position 407 [Y407T] of the second heavy chain. Alternative bi-specific antibodies of the present invention comprise amino acid changes to a tryptophan (W) at position 366 [T366W] of the first heavy chain and to a serine (S) at position 366 [T366S], an alanine (A) at position 368 [L368A] and a valine (V) at position 407 [Y407V] of the second heavy chain.

FIG. 2: Assessment of oligomerization state by analytical ultracentrifugation. A representative ZweiMab bispecific antibody as shown in FIG. 1 comprising the heavy chain and light chain pairs of SEQ ID NOs:23/24 and of SEQ ID NOs:25/26 respectively, and the linker of SEQ ID NO:7 in both chains was found be to >99% monomeric after two step purification. The peak at 5.64-6.78 S indicates monomers.

FIG. 3: SDS-PAGE: Integrity of a representative ZweiMab bispecific antibody as shown in FIG. 1 comprising the heavy chain and light chain pairs of SEQ ID NOs:23/24 and of SEQ ID NOs:27/28 respectively, and the linker of SEQ ID NO:7 in both chains was assessed by the SDS-PAGE gel electrophoresis (4-12% gradient gels). The estimated molecular weight of an intact bispecific antibody is ˜150 kDa, while the same sample under reducing condition (DTT 50 mM), is ˜75 kDa due to the reduction of disulfide bonds in the hinge region. Owing to a small molecular mass difference (Chain-A: 74,285 Da and Chain-B: 74,430 Da), the two chains are indistinguishable in SDS-PAGE (under reducing condition).

FIG. 4: The oligomerization state of a representative ZweiMab bispecific antibody as shown in FIG. 1 comprising the heavy chain and light chain pairs of SEQ ID NOs:27/28 and of SEQ ID NOs:29/30 respectively, and the linker of SEQ ID NO:7 in both chains was assessed by Analytic Size-exclusion chromatography (running buffer 50 mM Sodium Phosphate pH 6.5, 200 mM Arginine, 0.05% Sodium Azide, TSK3000). The sample of two-step purification was highly homogeneous (>99% monomeric).

FIGS. 5A and 5B: SPR binding assay shows that a representative ZweiMab bispecific antibody as shown in FIG. 1 comprising the heavy chain and light chain pairs of SEQ ID NOs:23/24 and of SEQ ID NOs:27/28 respectively, and the linker of SEQ ID NO:7 in both chains retains binding affinity towards the target antigen (human TNFα, FIG. 5A). As a control, one of the parental IgG (adalimumab) was also assessed for binding to human TNFα. The on-rate, off-rate as well as the equilibrium dissociation rate constant (K_{D}) values for the ZweiMab bispecific antibody is comparable to the parental IgG (FIG. 5B). The presence of linker between light and heavy chains appears to be non-interfering with the target antigen binding.

FIG. 6: Cynomolgus monkey PK study was performed using Male naïve Chinese cyno monkeys. A representative ZweiMab bispecific antibody comprising the heavy chain and light chain pairs of SEQ ID NOs:23/24 and of SEQ ID NOs:25/26 and the corresponding ZweiMab bispecific antibody with YTE mutations (comprising the heavy chain and light chain pairs of SEQ ID NOs:31/24 and of SEQ ID NOs:32/26 respectively), and the linker of SEQ ID NO:7 in the four chains were dosed at 0.6 mg/kg (IV bolus, single dose). The serum sampling for performed for 3-weeks post dosing.

FIG. 7A-B: Binding to Protein A in the absence or presence of H435Y/R436F mutations on the Fc domain tested in an ELISA-based method. A representative ZweiMab bispecific antibody (AB) comprising the heavy chain and light chain pairs of SEQ ID NOs:23/24 and of SEQ ID NOs:25/26 with H435Y/R436F mutations on the arm also comprising the Y407T mutation respectively, and the linker of SEQ ID NO:7 in both chains shows similar binding profile as a control IgG1. H435Y/R436F mutations on one-arm of the Fc led to weaker binding to Protein-A for one of homodimeric variant (AA), while the H435Y/R436F mutations on both the arms of the Fc led to significant loss in binding to Protein-A for the homodimeric variant (BB).

FIG. 8: Study of the linker length and composition. The polypeptide linker that connects the light and heavy chain in a ZweiMab bispecific antibody was engineered to vary in length (from 22 aa to 42 aa) and composition. The quality of the protein was assessed by analytical size exclusion chromatography.

### DETAILED DESCRIPTION

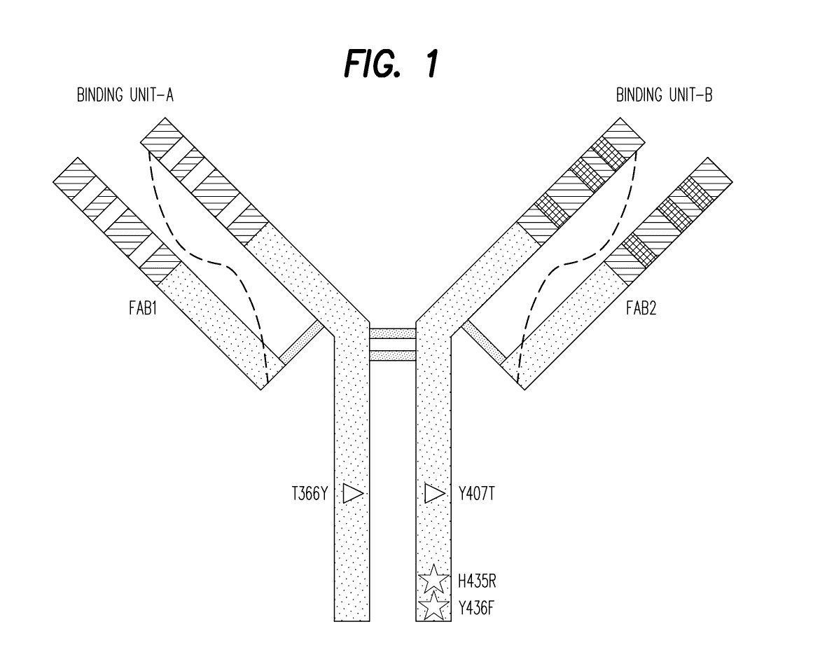

The present invention provides multi-specific binding proteins. The multi-specific binding proteins of the present invention provide a structure, into which binding units to target proteins are incorporated. The general structure of an exemplary multi-specific binding proteins of the present invention is depicted in FIG. 1 (in this case an examplary bi-specific binding protein), but multi-specific binding proteins are also encompassed in the present invention. The multi-specific binding proteins of the present invention are also referred to herein as “ZweiMab”, “ZweiMab antibodies” or “antibodies”. Some embodiments of ZweiMab are bi-specific and are referred herein in some instances as “ZweiMab bispecific antibodies” or “bispecific antibodies”. Some embodiments of ZweiMab are multi-specific and are referred herein in some instances as “ZweiMab multi-specific antibodies” or “multi-specific antibodies”.

In general, the multi-specific binding proteins of the present invention comprise at least two binding units that are specific to two different epitopes. In another aspect, the number of binding specificities in a protein of the present invention is increased by the addition of further binding units to the protein, thus resulting for example in tri-specific or quadri-specific binding protein, as for example described herein.

In one aspect, a protein of the present invention comprises a first heavy chain and a first light chain forming a first binding unit specific for a first epitope and a second heavy chain and a second light chain forming a second binding unit specific for a second epitope.

Generally, a heavy chain in a protein according to the present invention is derived from the heavy chain of an antibody, with the inclusion of amino acid changes as described below. Such heavy chain typically comprises at the amino-terminus a variable domain (V_{H}), followed by three constant domains (C_{H1}, C_{H2} and C_{H3}), as well as a hinge region between C_{H1} and C_{H2}. Generally, a light chain in a protein according to the present invention is derived from the light chain of an antibody. Such light chain typically comprises two domains, an amino-terminal variable domain (V_{L}) and a carboxy-terminal constant domain (C_{L}). Generally, the V_{L} domain associates non-covalently with the V_{H} domain, whereas the C_{L} domain is commonly covalently linked to the C_{H1} domain via a disulfide bond. Generally, the first and second heavy chains form a heterodimer through one or more di-sulfide bridges in a protein of the present invention. In the context of the present invention, a heavy chain is for example derived from the heavy chain of an IgG, for example an IgG₁, IgG₂ or IgG₄. For example, a heavy chain of the present invention is a heavy chain of an IgG₁ or IgG₄ and comprises a variable domain (V_{H}), followed by three constant domains (C_{H1}, C_{H2} and C_{H3}), as well as a hinge region between C_{H1} and C_{H2}. Examples of constant regions a heavy chain are shown in SEQ ID NO:1, 3-5, and 36-39. In the context of the present invention, a light chain is for example a kappa (κ) or a lambda (λ) light chain. In one aspect, such a light chain comprises two domains, an amino-terminal variable domain (V_{L}) and a carboxy-terminal constant domain (C_{L}). An example of a constant region of a kappa light chain is shown in SEQ ID NO:2. An example of a constant region of a lambda light chain is shown in SEQ ID NO:35.

The numbering of the amino acids in the amino acid chains of a protein of the present invention is herein according to the EU numbering system (Edelman, Cunningham et al. 1969), unless otherwise specified. This means that the amino acid numbers indicated herein correspond to the positions in a heavy chain of the corresponding sub-type (e.g. IgG₁ or IgG₄), according to the EU numbering system, unless otherwise specified.

In one aspect, the first heavy chain and said second heavy chain in a protein of the present invention each comprises one or more amino acid changes which reduce the formation of homodimers of the heavy chains. Through these changes, a “protrusion” is generated in one of the heavy chains by replacing one or more, small amino acid side chains from the interface of one of the heavy chains with larger side chains (e.g. tyrosine or tryptophan). Compensatory “cavities” of identical or similar size are created on the interface of the other heavy chain by replacing large amino acid side chains with smaller ones (e.g. alanine or threonine). This provides a mechanism for increasing the yield of the heterodimer over other unwanted end-products such as homodimers, in particular homodimers of the heavy chain with the “protrusion” (see for example Ridgway et al. Protein Eng, 1996. 9(7): p. 617-21). In one aspect, such amino acid changes are a tyrosine (Y) at position 366 [T366Y] of the first heavy chain and a threonine (T) at position 407 [Y407T] of the second heavy chain. In an alternative aspect, the first heavy chain comprises a serine (S) at position 366 [T366S] and the second heavy chain comprises a tryptophan (W) at position 366 [T366W], an alanine (A) at position 368 [L368A] and a valine (V) at position 407 [Y407V]. In an alternative aspect, the first heavy chain comprises a tryptophan (W) at position 366 [T366W] and the second heavy chain comprises a serine (S) at position 366 [T366S], an alanine (A) at position 368 [L368A] and a valine (V) at position 407 [Y407V]. In one aspect, such a heavy chain is a heavy chain derived from the heavy chain of an IgG₁ or IgG₄.

In one aspect, the first heavy chain comprises a cysteine (C) at position 354 [S354C] in addition to the tryptophan (W) at position 366 [T366W] and the second heavy chain comprises a cysteine (C) at position 349 [Y349C] in addition to the serine (S) at position 366 [T366S], the alanine (A) at position 368 [L368A] and the valine (V) at position 407 [Y407V]. In one aspect, such a heavy chain is a heavy chain derived from the heavy chain of an IgG₄.

The amino acid changes above, for example the amino acid changes at position 366 [T366Y] of the first heavy chain and at position 407 [Y407T] of the second heavy chain, have the additional benefit of low immunogenicity.

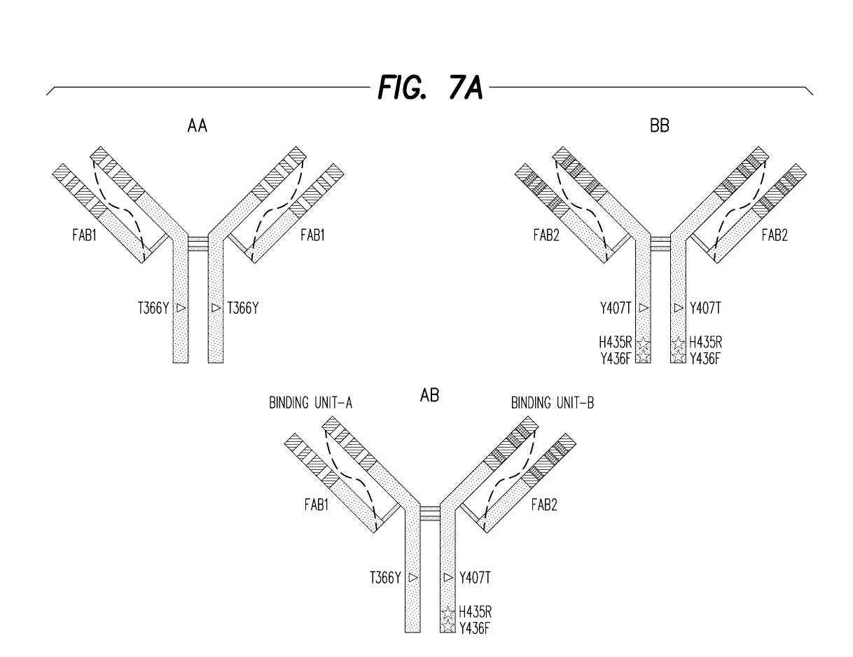

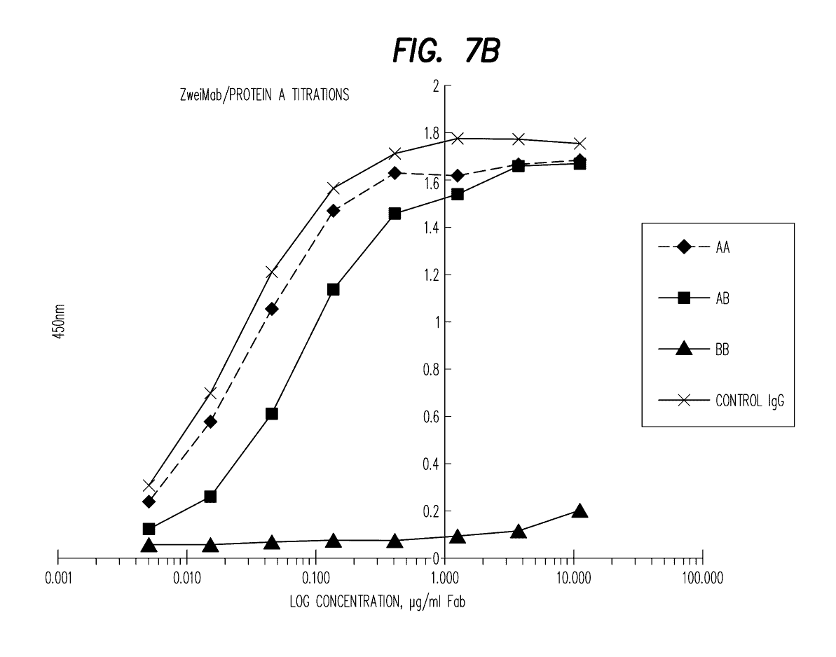

In a further aspect, the first heavy chain or the second heavy chain in a protein of the present invention further comprises one or more amino acid changes which reduce the binding of the heavy chain to protein A. In one aspect, such amino acid changes are an arginine at position 435 [H435R] and a phenylalanine at position 436 [Y436F] of one of the heavy chains. Both changes are derived from the sequence of human IgG3 (IgG3 does not bind to protein A). These two mutations are located in the CH3 domain and are incorporated in one of the heavy chains to reduce binding to Protein A (see for example Jendeberg et al. J Immunol Methods, 1997. 201(1): p. 25-34). These two changes facilitate the removal of homodimers of heavy chains comprising these changes during protein purification (see for example FIG. 7A-B).

In one aspect, in a protein of the present invention, the heavy chain, which comprises a threonine (T) at position 407 [Y407T], further comprises an arginine at position 435 [H435R] and a phenylalanine at position 436 [Y436F]. In this case, the other heavy chain comprises a tyrosine (Y) at position 366 [T366Y], but does not include the two changes at positions 435 and 436. This is shown for example in FIG. 1. Alternatively, in one aspect, in a protein of the present invention, the heavy chain, which comprises a serin (S) at position 366 [T366S], an alanine (A) at position 368 [L368A] and a valine (V) at position 407 [Y407V], further comprises an arginine at position 435 [H435R] and a phenylalanine at position 436 [Y436F]. In this case, the other heavy chain comprises a tryptophan (W) at position 366 [T366W], but does not include the two changes at positions 435 and 436. Thus, the heavy chain comprising the amino acid change resulting in a “cavity” as described above also comprises the amino acid changes, which reduce binding to Protein A. Homodimers comprising this heavy chain are removed through reduced binding to Protein A. The production of homodimers of the other heavy chain, which comprises the “protrusion”, is reduced by the presence of the “protrusion”.

In a further aspect, in a protein if the present invention, the heavy chain and the light chain in one of the binding units are covalently linked through a linker. In a further aspect, the heavy chain and the light chain in the two binding units are respectively covalently linked through a linker. This averts mis-pairing of the light chains during expression and purification of proteins, and allows the use of a wide variety of light chains in a protein of the present invention without compromising the functionality and/or bind affinity of the binding units.

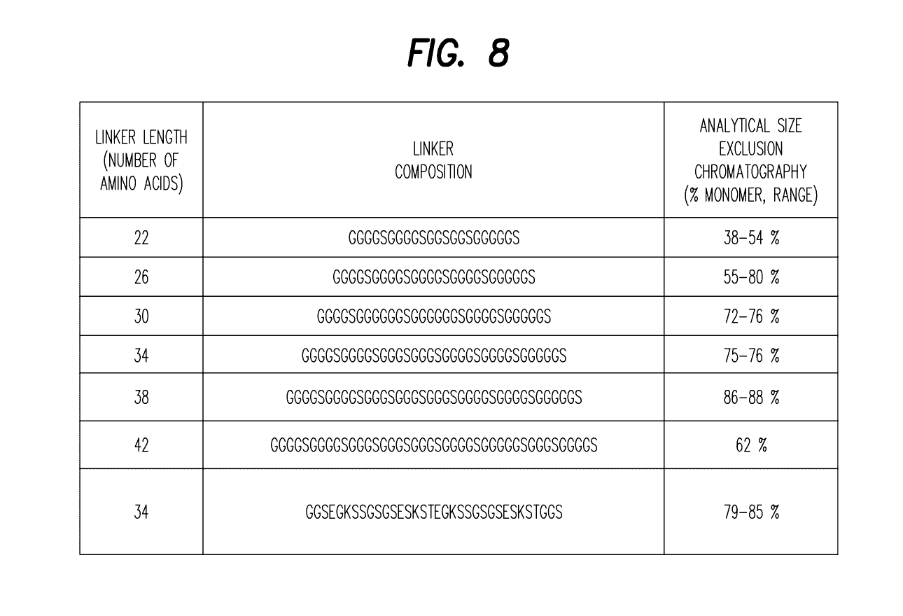

In one aspect, the first linker is covalently linked to the N-terminus of the first heavy chain and to the C-terminus of the first light chain and the second linker is covalently linked to the N-terminus of the second heavy chain and to the C-terminus of the second light chain. In one aspect, a linker used in a protein of the present invention comprises 26 to 42 amino acids, for example 30 to 40 amino acids. In a further aspect, a linker used in a protein of the present invention comprises 34 to 40 amino acids, for example 36 to 39 amino acids, for example 38 amino acids. In one aspect, the first and said second linkers have the same length. In one aspect, the first and said second linkers have different length. In one aspect, the first and said second linker are identical. In one aspect, the first and said second linker have different sequence composition. Representative examples of linkers used in a protein of the present invention are shown Table 2 and FIG. 8 herein.

In a further aspect, the Fc domain of a protein of the present invention may or may not further comprises YTE mutations (M252Y/S254T/T256E, EU numbering (Dall'Acqua, Kiener et al. 2006)). These mutations have been shown to improve the pharmacokinetic properties of Fc domains through preferential enhancement of binding affinity for neonatal FcRn receptor at pH 6.0.

In a further aspect, a heavy chain of the present invention derived from an IgG1 also includes the “KO” mutations (L234A, L235A). In a further aspect, a heavy chain of the present invention derived from an IgG4 also includes the Pro hinge mutation (S228P).

Accordingly, in one aspect, the present invention provides a protein comprising a first amino acid chain and a second amino acid chain, wherein the first chain comprises a first light chain covalently linked to a linker, which is itself covalently linked to a first heavy chain, and wherein the second chain comprises a second light chain covalently linked to a linker, which is itself covalently linked to a second heavy chain.

In one aspect, starting from its N-terminus, the first chain comprises a light chain variable region, a light chain constant region, a linker, a heavy chain variable region and a heavy chain constant region. In one aspect, starting from its N-terminus, the second chain comprises a light chain variable region, a light chain constant region, a linker, a heavy chain variable region and a heavy chain constant region. In one aspect, both the first and the second chains comprise starting from their N-terminus a light chain variable region, a light chain constant region, a linker, a heavy chain variable region and a heavy chain constant region.

The resulting proteins bears a full Fc, which is marginally larger than an IgG and has two independent binding sites, each for one target protein or for an epitope on a target protein. This format greatly reduces heterogeneity after expression and purification and maintains the functional properties of the binding moieties. This also enables the expression of homogenous proteins, which express well, e.g. in mammalian cells. The proteins of the present invention have an acceptable immunogenicity profile and have satisfactory stability in-vitro and in-vivo.

The multi-specific binding proteins of the present invention comprise at least two binding units that are specific to two different epitopes. In one aspect, the two epitopes are epitopes of two different target proteins. In another aspect, the two epitopes are epitopes of the same target protein. For example, binding to multiple target proteins, such as targets that are present in a complex, or targets for which sequestering and/or clustering, can increase the therapeutic properties of a binding protein. Alternatively, binding to more than one epitope of the same target protein may confer greater specificity than a mono-specific protein that binds to only one epitope on a target protein.

Epitopes are most commonly proteins, short peptides, or combinations thereof. The minimum size of a peptide or polypeptide epitope is thought to be about four to five amino acids. Peptide or polypeptide epitopes contain for example at least seven amino acids or for example at least nine amino acids or for example between about 15 to about 20 amino acids. Since a binding unit can recognize an antigenic peptide or polypeptide in its tertiary form, the amino acids comprising an epitope need not be contiguous, and in some cases, may not even be on the same peptide chain. Epitopes may be determined by various techniques known in the art, such as X-ray crystallography, nuclear magnetic resonance, Hydrogen/Deuterium Exchange Mass Spectrometry (HXMS), site-directed mutagenesis, alanine scanning mutagenesis, and peptide screening methods.

Pairs of target proteins recognized by binding units according to the present invention may be in the same biochemical pathway or in different pathways.

In one aspect, a binding unit of a binding protein according to the present invention comprises a heavy chain variable domain (V_{H}) and a light chain variable domain (V_{L}) derived from an antibody. Such variable domain may be optimized variable domain as described herein. In such case, each variable domain comprises 3 CDRs as described herein. In one aspect, a binding protein according to the present invention or certain portions of the protein is generally derived from an antibody. The generalized structure of antibodies or immunoglobulin is well known to those of skill in the art. These molecules are heterotetrameric glycoproteins, typically of about 150,000 daltons, composed of two identical light (L) chains and two identical heavy (H) chains and are typically referred to as full length antibodies. Each light chain is covalently linked to a heavy chain by one disulfide bond to form a heterodimer, and the heterotetrameric molecule is formed through a covalent disulfide linkage between the two identical heavy chains of the heterodimers. Although the light and heavy chains are linked together by one disulfide bond, the number of disulfide linkages between the two heavy chains varies by immunoglobulin isotype. Each heavy and light chain also has regularly spaced intrachain disulfide bridges. Each heavy chain has at the amino-terminus a variable domain (V_{H}), followed by three or four constant domains (C_{H1}, C_{H2}, C_{H3}, and C_{H4}), as well as a hinge region between C_{H1} and C_{H2}. Each light chain has two domains, an amino-terminal variable domain (V_{L}) and a carboxy-terminal constant domain (C_{L}). The V_{L} domain associates non-covalently with the V_{H} domain, whereas the C_{L} domain is commonly covalently linked to the C_{H1} domain via a disulfide bond. Particular amino acid residues are believed to form an interface between the light and heavy chain variable domains (Chothia et al., 1985, J. Mol. Biol. 186:651-663). Variable domains are also referred herein as variable regions.

Certain domains within the variable domains differ between different antibodies i.e., are “hypervariable.” These hypervariable domains contain residues that are directly involved in the binding and specificity of each particular antibody for its specific antigenic determinant. Hypervariability, both in the light chain and the heavy chain variable domains, is concentrated in three segments known as complementarity determining regions (CDRs) or hypervariable loops (HVLs). CDRs are defined by sequence comparison in Kabat et al., 1991, in: Sequences of Proteins of Immunological Interest, 5^{th} Ed. Public Health Service, National Institutes of Health, Bethesda, Md., whereas HVLs (also referred herein as CDRs) are structurally defined according to the three-dimensional structure of the variable domain, as described by Chothia and Lesk, 1987, J. Mol. Biol. 196: 901-917. These two methods result in slightly different identifications of a CDR. As defined by Kabat, CDR-L1 is positioned at about residues 24-34, CDR-L2, at about residues 50-56, and CDR-L3, at about residues 89-97 in the light chain variable domain; CDR-H1 is positioned at about residues 31-35, CDR-H2 at about residues 50-65, and CDR-H3 at about residues 95-102 in the heavy chain variable domain. The exact residue numbers that encompass a particular CDR will vary depending on the sequence and size of the CDR. Those skilled in the art can routinely determine which residues comprise a particular CDR given the variable region amino acid sequence of the antibody. The CDR1, CDR2, CDR3 of the heavy and light chains therefore define the unique and functional properties specific for a given antibody.

The three CDRs within each of the heavy and light chains are separated by framework regions (FR), which contain sequences that tend to be less variable. From the amino terminus to the carboxy terminus of the heavy and light chain variable domains, the FRs and CDRs are arranged in the order: FR1, CDR1, FR2, CDR2, FR3, CDR3, and FR4. The largely β-sheet configuration of the FRs brings the CDRs within each of the chains into close proximity to each other as well as to the CDRs from the other chain. The resulting conformation contributes to the antigen binding site (see Kabat et al., 1991, NIH Publ. No. 91-3242, Vol. I, pages 647-669), although not all CDR residues are necessarily directly involved in antigen binding.

FR residues and Ig constant domains are not directly involved in antigen binding, but contribute to antigen binding and/or mediate antibody effector function. Some FR residues are thought to have a significant effect on antigen binding in at least three ways: by noncovalently binding directly to an epitope, by interacting with one or more CDR residues, and by affecting the interface between the heavy and light chains. The constant domains are not directly involved in antigen binding but mediate various Ig effector functions, such as participation of the antibody in antibody dependent cellular cytotoxicity (ADCC), complement dependent cytotoxicity (CDC) and antibody dependent cellular phagocytosis (ADCP).

The light chains of vertebrate immunoglobulins are assigned to one of two clearly distinct classes, kappa (κ) and lambda (λ), based on the amino acid sequence of the constant domain. By comparison, the heavy chains of mammalian immunoglobulins are assigned to one of five major classes, according to the sequence of the constant domains: IgA, IgD, IgE, IgG, and IgM. IgG and IgA are further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively. The subunit structures and three-dimensional configurations of the classes of native immunoglobulins are well known.

In some embodiments, a binding protein of the present invention includes a constant region that mediates effector function. The constant region can provide antibody-dependent cellular cytotoxicity (ADCC), antibody-dependent cellular phagocytosis (ADCP) and/or complement-dependent cytotoxicity (CDC) responses. The effector domain(s) can be, for example, an Fc region of an Ig molecule.

The effector domain of an antibody can be from any suitable vertebrate animal species and isotypes. The isotypes from different animal species differ in the abilities to mediate effector functions. For example, the ability of human immunoglobulin to mediate CDC and ADCC/ADCP is generally in the order of IgM≈-IgG₁≈IgG₃>IgG₂>IgG₄ and IgG₁≈IgG₃>IgG₂/IgM/IgG₄, respectively. Murine immunoglobulins mediate CDC and ADCC/ADCP generally in the order of murine IgM≈IgG₃>>IgG_{2b}>IgG₂ₐ>>IgG₁ and IgG_{2b}>IgG₂ₐ>IgG₁>>IgG₃, respectively. In another example, murine IgG₂ₐ mediates ADCC while both murine IgG₂ₐ and IgM mediate CDC.

The term “antibody” encompasses monoclonal antibodies (including full length monoclonal antibodies), polyclonal antibodies, multispecific antibodies (e.g., bispecific antibodies), and antibody fragments such as variable domains and other portions of antibodies that exhibit one or more desired biological activity/ies.

The term “monomer” refers to a homogenous form of an antibody. For example, for a full-length antibody, monomer means a monomeric antibody having two identical heavy chains and two identical light chains. In the context of the present invention, a monomer means a protein of the present invention having two heavy chains and two light chains as described herein.

The term “antibody fragment” refers to a portion of a full length antibody, in which a variable region or a functional capability is retained. Examples of antibody fragments include, but are not limited to, a Fab, Fab′, F(ab′)₂, Fd, Fv, scFv and scFv-Fc fragment.

Full length antibodies can be treated with enzymes such as papain or pepsin to generate useful antibody fragments. Papain digestion is used to produces two identical antigen-binding antibody fragments called “Fab” fragments, each with a single antigen-binding site, and a residual “Fc” fragment. The Fab fragment also contains the constant domain of the light chain and the C_{H1} domain of the heavy chain. Pepsin treatment yields a F(ab′)₂ fragment that has two antigen-binding sites and is still capable of cross-linking antigen.

Fab′ fragments differ from Fab fragments by the presence of additional residues including one or more cysteines from the antibody hinge region at the C-terminus of the C_{H1} domain. F(ab′)₂ antibody fragments are pairs of Fab′ fragments linked by cysteine residues in the hinge region. Other chemical couplings of antibody fragments are also known.

“Fv” fragment contains a complete antigen-recognition and binding site consisting of a dimer of one heavy and one light chain variable domain in tight, non-covalent association. In this configuration, the three CDRs of each variable domain interact to define an antigen-biding site on the surface of the V_{H}-V_{L} dimer. Collectively, the six CDRs confer antigen-binding specificity to the antibody.

A “single-chain Fv” or “scFv” antibody fragment is a single chain Fv variant comprising the V_{H} and V_{L} domains of an antibody where the domains are present in a single polypeptide chain. The single chain Fv is capable of recognizing and binding antigen. The scFv polypeptide may optionally also contain a polypeptide linker positioned between the V_{H} and V_{L} domains in order to facilitate formation of a desired three-dimensional structure for antigen binding by the scFv (see, e.g., Pluckthun, 1994, In The Pharmacology of monoclonal Antibodies, Vol. 113, Rosenburg and Moore eds., Springer-Verlag, New York, pp. 269-315).

An “optimized antibody” or an “optimized antibody fragment” is a specific type of chimeric antibody which includes an immunoglobulin amino acid sequence variant, or fragment thereof, which is capable of binding to a predetermined antigen and which, comprises one or more FRs having substantially the amino acid sequence of a human immunoglobulin and one or more CDRs having substantially the amino acid sequence of a non-human immunoglobulin. This non-human amino acid sequence often referred to as an “import” sequence is typically taken from an “import” antibody domain, particularly a variable domain. In general, an optimized antibody includes at least the CDRs or HVLs of a non-human antibody or derived from a non-human antibody, inserted between the FRs of a human heavy or light chain variable domain. It will be understood that certain mouse FR residues may be important to the function of the optimized antibodies and therefore certain of the human germline sequence heavy and light chain variable domains residues are modified to be the same as those of the corresponding mouse sequence. During this process undesired amino acids may also be removed or changed, for example to avoid deamidation, undesirable charges or lipophilicity or non-specific binding. An “optimized antibody”, an “optimized antibody fragment” or “optimized” may sometimes be referred to as “humanized antibody”, “humanized antibody fragment” or “humanized”, or as “sequence-optimized”.

Immunoglobulin residues that affect the interface between heavy and light chain variable regions (“the V_{L}-V_{H} interface”) are those that affect the proximity or orientation of the two chains with respect to one another. Certain residues that may be involved in interchain interactions include V_{L} residues 34, 36, 38, 44, 46, 87, 89, 91, 96, and 98 and V_{H} residues 35, 37, 39, 45, 47, 91, 93, 95, 100, and 103 (utilizing the numbering system set forth in Kabat et al., Sequences of Proteins of Immunological Interest (National Institutes of Health, Bethesda, Md., 1987)). U.S. Pat. No. 6,407,213 also discusses that residues such as V_{L} residues 43 and 85, and V_{H} residues 43 and 60 also may be involved in this interaction. While these residues are indicated for human IgG only, they are applicable across species. Important antibody residues that are reasonably expected to be involved in interchain interactions are selected for substitution into the consensus sequence.

The terms “consensus sequence” and “consensus antibody” refer to an amino acid sequence which comprises the most frequently occurring amino acid residue at each location in all immunoglobulins of any particular class, isotype, or subunit structure, e.g., a human immunoglobulin variable domain. The consensus sequence may be based on immunoglobulins of a particular species or of many species. A “consensus” sequence, structure, or antibody is understood to encompass a consensus human sequence as described in certain embodiments, and to refer to an amino acid sequence which comprises the most frequently occurring amino acid residues at each location in all human immunoglobulins of any particular class, isotype, or subunit structure. Thus, the consensus sequence contains an amino acid sequence having at each position an amino acid that is present in one or more known immunoglobulins, but which may not exactly duplicate the entire amino acid sequence of any single immunoglobulin. The variable region consensus sequence is not obtained from any naturally produced antibody or immunoglobulin. Kabat et al., 1991, Sequences of Proteins of Immunological Interest, 5th Ed. Public Health Service, National Institutes of Health, Bethesda, Md., and variants thereof. The FRs of heavy and light chain consensus sequences, and variants thereof, provide useful sequences for the preparation of antibodies. See, for example, U.S. Pat. Nos. 6,037,454 and 6,054,297.

Human germline sequences are found naturally in the human population. A combination of those germline genes generates antibody diversity. Germline antibody sequences for the light chain of the antibody come from conserved human germline kappa or lambda v-genes and j-genes. Similarly the heavy chain sequences come from germline v-, d- and j-genes (LeFranc, M-P, and LeFranc, G, “The Immunoglobulin Facts Book” Academic Press, 2001).

An “isolated” antibody is one that has been identified and separated and/or recovered from a component of its natural environment. Contaminant components of the antibody's natural environment are those materials that may interfere with diagnostic or therapeutic uses of the antibody, and can be enzymes, hormones, or other proteinaceous or nonproteinaceous solutes. In one aspect, the antibody will be purified to at least greater than 95% isolation by weight of antibody.

An isolated antibody includes an antibody in situ within recombinant cells in which it is produced, since at least one component of the antibody's natural environment will not be present. Ordinarily however, an isolated antibody will be prepared by at least one purification step in which the recombinant cellular material is removed.

The term “antibody performance” refers to factors that contribute to antibody recognition of antigen or the effectiveness of an antibody in vivo. Changes in the amino acid sequence of an antibody can affect antibody properties such as folding, and can influence physical factors such as initial rate of antibody binding to antigen (kₐ), dissociation constant of the antibody from antigen (k_{d}), affinity constant of the antibody for the antigen (Kd), non-specific binding, conformation of the antibody, protein stability, and half life of the antibody.

The term “epitope tagged” when used herein, refers to an antibody fused to an “epitope tag”. An “epitope tag” is a polypeptide having a sufficient number of amino acids to provide an epitope for antibody production, yet is designed such that it does not interfere with the desired activity of the antibody. The epitope tag is usually sufficiently unique such that an antibody raised against the epitope tag does not substantially cross-react with other epitopes. Suitable tag polypeptides generally contain at least 6 amino acid residues and usually contain about 8 to 50 amino acid residues, or about 9 to 30 residues. Examples of epitope tags and the antibody that binds the epitope include the flu HA tag polypeptide and its antibody 12CA5 (Field et al., 1988 Mol. Cell. Biol. 8: 2159-2165; c-myc tag and 8F9, 3C7, 6E10, G4, B7 and 9E10 antibodies thereto (Evan et al., 1985, Mol. Cell. Biol. 5(12):3610-3616; and Herpes simplex virus glycoprotein D (gD) tag and its antibody (Paborsky et al. 1990, Protein Engineering 3(6): 547-553). In certain embodiments, the epitope tag is a “salvage receptor binding epitope”. As used herein, the term “salvage receptor binding epitope” refers to an epitope of the Fc region of an IgG molecule (such as IgG₁, IgG₂, IgG₃, or IgG₄) that is responsible for increasing the in vivo serum half-life of the IgG molecule.

In some embodiments, the antibodies of the present invention may be conjugated to a cytotoxic agent. This is any substance that inhibits or prevents the function of cells and/or causes destruction of cells. The term is intended to include radioactive isotopes (such as I¹³¹, I¹²⁵, Y⁹⁰, and Re¹⁸⁶), chemotherapeutic agents, and toxins such as enzymatically active toxins of bacterial, fungal, plant, or animal origin, and fragments thereof. Such cytotoxic agents can be coupled to the antibodies of the present invention using standard procedures, and used, for example, to treat a patient indicated for therapy with the antibody.

A “chemotherapeutic agent” is a chemical compound useful in the treatment of cancer. There are numerous examples of chemotherapeutic agents that could be conjugated with the therapeutic antibodies of the present invention.

The antibodies also may be conjugated to prodrugs. A “prodrug” is a precursor or derivative form of a pharmaceutically active substance that is less cytotoxic to tumor cells compared to the parent drug and is capable of being enzymatically activated or converted into the more active form. See, for example, Wilman, 1986, “Prodrugs in Cancer Chemotherapy”, In Biochemical Society Transactions, 14, pp. 375-382, 615th Meeting Belfast and Stella et al., 1985, “Prodrugs: A Chemical Approach to Targeted Drug Delivery, In: “Directed Drug Delivery, Borchardt et al., (ed.), pp. 247-267, Humana Press. Useful prodrugs include, but are not limited to, phosphate-containing prodrugs, thiophosphate-containing prodrugs, sulfate-containing prodrugs peptide-containing prodrugs, D-amino acid-modified prodrugs, glycosylated prodrugs, β-lactam-containing prodrugs, optionally substituted phenoxyacetamide-containing prodrugs, and optionally substituted phenylacetamide-containing prodrugs, 5-fluorocytosine and other 5-fluorouridine prodrugs that can be converted into the more active cytotoxic free drug. Examples of cytotoxic drugs that can be derivatized into a prodrug form include, but are not limited to, those chemotherapeutic agents described above.

For diagnostic as well as therapeutic monitoring purposes, the antibodies of the invention also may be conjugated to a label, either a label alone or a label and an additional second agent (prodrug, chemotherapeutic agent and the like). A label, as distinguished from the other second agents refers to an agent that is a detectable compound or composition and it may be conjugated directly or indirectly to an antibody of the present invention. The label may itself be detectable (e.g., radioisotope labels or fluorescent labels) or, in the case of an enzymatic label, may catalyze chemical alteration of a substrate compound or composition that is detectable. Labeled antibody can be prepared and used in various applications including in vitro and in vivo diagnostics.

The antibodies of the present invention may be formulated as part of a liposomal preparation in order to affect delivery thereof in vivo. A “liposome” is a small vesicle composed of various types of lipids, phospholipids, and/or surfactant. Liposomes are useful for delivery to a mammal of a compound or formulation, such as an antibody disclosed herein, optionally, coupled to or in combination with one or more pharmaceutically active agents and/or labels. The components of the liposome are commonly arranged in a bilayer formation, similar to the lipid arrangement of biological membranes.

Certain aspects of the present invention relate to isolated nucleic acids that encode one or more domains of the antibodies of the present invention, for example antibodies of the present invention. An “isolated” nucleic acid molecule is a nucleic acid molecule that is identified and separated from at least one contaminant nucleic acid molecule with which it is ordinarily associated in the natural source of the antibody nucleic acid. An isolated nucleic acid molecule is distinguished from the nucleic acid molecule as it exists in natural cells.

In various aspects of the present invention one or more domains of the antibodies will be expressed in a recombinant form. Such recombinant expression may employ one or more control sequences, i.e., polynucleotide sequences necessary for expression of an operably linked coding sequence in a particular host organism. The control sequences suitable for use in prokaryotic cells include, for example, promoter, operator, and ribosome binding site sequences. Eukaryotic control sequences include, but are not limited to, promoters, polyadenylation signals, and enhancers. These control sequences can be utilized for expression and production of antibody in prokaryotic and eukaryotic host cells.

A nucleic acid sequence is “operably linked” when it is placed into a functional relationship with another nucleic acid sequence. For example, a nucleic acid presequence or secretory leader is operably linked to a nucleic acid encoding a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, enhancers are optionally contiguous. Linking can be accomplished by ligation at convenient restriction sites. If such sites do not exist, synthetic oligonucleotide adaptors or linkers can be used.

As used herein, the expressions “cell”, “cell line”, and “cell culture” are used interchangeably and all such designations include the progeny thereof. Thus, “transformants” and “transformed cells” include the primary subject cell and cultures derived therefrom without regard for the number of transfers.

The term “mammal” for purposes of treatment refers to any animal classified as a mammal, including humans, domesticated and farm animals, and zoo, sports, or pet animals, such as dogs, horses, cats, cows, and the like. Preferably, the mammal is human.

A “disorder”, as used herein, is any condition that would benefit from treatment with an antibody described herein. This includes chronic and acute disorders or diseases including those pathological conditions that predispose the mammal to the disorder in question. Non-limiting examples of disorders to be treated herein include inflammatory, angiogenic, autoimmune and immunologic disorders, respiratory disorders, central nervous system disorders, eye disorders, cardiovascular disorders, cancer, hematological malignancies, benign and malignant tumors, leukemias and lymphoid malignancies.

The terms “cancer” and “cancerous” refer to or describe the physiological condition in mammals that is typically characterized by unregulated cell growth. Examples of cancer include, but are not limited to carcinoma, lymphoma, blastoma, sarcoma, and leukemia.

The term “intravenous infusion” refers to introduction of an agent into the vein of an animal or human patient over a period of time greater than approximately 15 minutes, generally between approximately 30 to 90 minutes.

The term “intravenous bolus” or “intravenous push” refers to drug administration into a vein of an animal or human such that the body receives the drug in approximately 15 minutes or less, generally 5 minutes or less.

The term “subcutaneous administration” refers to introduction of an agent under the skin of an animal or human patient, preferable within a pocket between the skin and underlying tissue, by relatively slow, sustained delivery from a drug receptacle.

Pinching or drawing the skin up and away from underlying tissue may create the pocket.

The term “subcutaneous infusion” refers to introduction of a drug under the skin of an animal or human patient, preferably within a pocket between the skin and underlying tissue, by relatively slow, sustained delivery from a drug receptacle for a period of time including, but not limited to, 30 minutes or less, or 90 minutes or less. Optionally, the infusion may be made by subcutaneous implantation of a drug delivery pump implanted under the skin of the animal or human patient, wherein the pump delivers a predetermined amount of drug for a predetermined period of time, such as 30 minutes, 90 minutes, or a time period spanning the length of the treatment regimen.

The term “subcutaneous bolus” refers to drug administration beneath the skin of an animal or human patient, where bolus drug delivery is less than approximately 15 minutes; in another aspect, less than 5 minutes, and in still another aspect, less than 60 seconds. In yet even another aspect, administration is within a pocket between the skin and underlying tissue, where the pocket may be created by pinching or drawing the skin up and away from underlying tissue.

The term “therapeutically effective amount” is used to refer to an amount of an active agent that relieves or ameliorates one or more of the symptoms of the disorder being treated. In another aspect, the therapeutically effective amount refers to a target serum concentration that has been shown to be effective in, for example, slowing disease progression. Efficacy can be measured in conventional ways, depending on the condition to be treated.

The terms “treatment” and “therapy” and the like, as used herein, are meant to include therapeutic as well as prophylactic, or suppressive measures for a disease or disorder leading to any clinically desirable or beneficial effect, including but not limited to alleviation or relief of one or more symptoms, regression, slowing or cessation of progression of the disease or disorder. Thus, for example, the term treatment includes the administration of an agent prior to or following the onset of a symptom of a disease or disorder thereby preventing or removing one or more signs of the disease or disorder. As another example, the term includes the administration of an agent after clinical manifestation of the disease to combat the symptoms of the disease. Further, administration of an agent after onset and after clinical symptoms have developed where administration affects clinical parameters of the disease or disorder, such as the degree of tissue injury or the amount or extent of metastasis, whether or not the treatment leads to amelioration of the disease, comprises “treatment” or “therapy” as used herein. Moreover, as long as the compositions of the invention either alone or in combination with another therapeutic agent alleviate or ameliorate at least one symptom of a disorder being treated as compared to that symptom in the absence of use of the antibody composition, the result should be considered an effective treatment of the underlying disorder regardless of whether all the symptoms of the disorder are alleviated or not.

The term “package insert” is used to refer to instructions customarily included in commercial packages of therapeutic products, that contain information about the indications, usage, administration, contraindications and/or warnings concerning the use of such therapeutic products.

Representative heavy chains constant regions and light chains constant regions of a binding protein according to the present invention are shown in Table 1. In a binding protein of the present invention, variable regions are linked to the constant regions at the N-terminus of the constant regions. Residues T366Y, Y407T, H435R, Y436F, and YTE-mutations as described herein are shown in bold and underlined in Table 1. Residues T366W, T366S, L368A and Y407V are also shown in bold and underlined in Table 1.

The heavy chain constant regions in SEQ ID NOs:1, 3-5, 36 and 38 are derived from an IgG1. The heavy chains in SEQ ID NOs:36 and 38 also include the “KO” mutations (L234A, L235A, in bold and underlined).

The heavy chain constant regions in SEQ ID NOs:37 and 39 are derived from an IgG4 and also include the Pro hinge mutation (S228P, in bold and underlined).

The light chain constant region in SEQ ID NO:2 is a kappa chain. The light chain constant region in SEQ ID NO:35 is a lambda chain (lambda 6 sub-type).

Table 2 shows representative linkers used in the binding proteins of the present invention.

Table 3 shows representative light chain variable regions and heavy chain variable regions used in a binding protein of the present invention.

Table 4 shows representative light chains and heavy chains of a binding protein of the present invention with different combinations of variable regions and constant regions. Table 4 also shows representative examples of amino acid chains comprised in a protein of the present invention.

In SEQ ID NOs:49-56, the amino acid sequences underlined are light chain variable regions and heavy chain variable regions, and the amino acid sequences in bold are linker sequences. Certain amino acids in heavy chains are shown as bold and underlined.

TABLE 1
Amino Acid Sequences

| | |
|---|---|
| heavy chain | ASTKGPSVFPLAPSSKSTSGGTAALGCLVKDYFPEPVTVSWNSGALT |
| constant | SGVHTFPAVLQSSGLYSLSSVVTVPSSSLGTQTYICNVNHKPSNTKV |
| region | DKRVEPKSCDKTHTCPPCPAPELLGGPSVFLFPPKPKDTLMISRTPE |
| | VTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLP |
| | PSREEMTKNQVSLYCLVKGFYPSDIAVEWESNGQPENNYKTTPPVLD |
| | SDGSFFLYSKLTVDKSRWQQGNVFSCSVMHEALHNHYTQKSLSLSPG |
| | K (SEQ ID NO: 1) |
| light chain | RTVAAPSVFIFPPSDEQLKSGTASVVCLLNNFYPREAKVQWKVDNAL |
| constant | QSGNSQESVTEQDSKDSTYSLSSTLTLSKADYEKHKVYACEVTHQGL |
| region | SSPVTKSFNRGEC (SEQ ID NO: 2) |
| heavy chain | ASTKGPSVFPLAPSSKSTSGGTAALGCLVKDYFPEPVTVSWNSGALT |
| constant | SGVHTFPAVLQSSGLYSLSSVVTVPSSSLGTQTYICNVNHKPSNTKV |
| region | DKRVEPKSCDKTHTCPPCPAPELLGGPSVFLFPPKPKDTLMISRTPE |
| | VTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLP |
| | PSREEMTKNQVSLTCLVKGFYPSDIAVEWESNGQPENNYKTTPPVLD |
| | SDGSFFLTSKLTVDKSRWQQGNVFSCSVMHEALHNRFTQKSLSLSPG |
| | K (SEQ TD NO: 3) |
| heavy chain | ASTKGPSVFPLAPSSKSTSGGTAALGCLVKDYFPEPVTVSWNSGALT |
| constant | SGVHTFPAVLQSSGLYSLSSVVTVPSSSLGTQTYICNVNHKPSNTKV |
| region with | DKRVEPKSCDKTHTCPPCPAPELLGGPSVFLFPPKPKDTLYITREPE |
| YTE | VTCVVVDVSHEDPEVKFNWYVDGVEVHNAKTKPREEQYNSTYRVVSV |
| mutations | LTVLHQDWLNGKEYKCKVSNKALPAPIEKTISKAKGQPREPQVYTLP |
| | PSREEMTKNQVSLYCLVKGFYPSDIAVEWESNGQPENNYKTTPPVLD |
| | SDGSFFLYSKLTVDKSRWQQGNVFSCSVMHEALHNHYTQKSLSLSPG |
| | K (SEQ ID NO: 4) |
| heavy chain | ASTKGPSVFPLAPSSKSTSGGTAALGCLVKDYFPEPVTVSWNSGALT |
| constant | SGVHTFPAVLQSSGLYSLSSVVTVPSSSLGTQTYICNVNHKPSNTKV |
| region with | DKRVEPKSCDKTHTCPPCPAPELLGGPSVFLFPPKPKDTLYITREPE |
| YTE | VTCVVVDVSHEDPEVKFNWYVDGVEVHNAKTKPREEQYNSTYRVVSV |
| mutations | LTVLHQDWLNGKEYKCKVSNKALPAPIEKTISKAKGQPREPQVYTLP |
| | PSREEMTKNQVSLTCLVKGFYPSDIAVEWESNGQPENNYKTTPPVLD |
| | SDGSFFLTSKLTVDKSRWQQGNVFSCSVMHEALHNRFTQKSLSLSPG |
| | K (SEQ ID NO: 5) |
| light | GQPKAAPSVTLFPPSSEELQANKATLVCLISDFYPGAVKVAWKADGS |
| chain | PVNTGVETTTPSKQSNNKYAASSYLSLTPEQWKSHRSYSCQVTHEGS |
| constant | TVEKTVAPAECS (SEQ ID NO: 35) |
| region | |
| heavy | ASTKGPSVFPLAPSSKSTSGGTAALGCLVKDYFPEPVTVSWNSGALT |
| chain | SGVHTFPAVLQSSGLYSLSSVVTVPSSSLGTQTYICNVNHKPSNTKV |
| constant | DKRVEPKSCDKTHTCPPCPAPEAAGGPSVFLFPPKPKDTLMISRTPE |
| region | VTCVVVDVSHEDPEVKFNWYVDGVEVHNAKTKPREEQYNSTYRVVSV |
| (IgG1) | LTVLHQDWLNGKEYKCKVSNKALPAPIEKTISKAKGQPREPQVYTLP |
| | PSREEMTKNQVSLWCLVKGFYPSDIAVEWESNGQPENNYKTTPPVLD |
| | SDGSFFLYSKLTVDKSRWQQGNVFSCSVMHEALHNHYTQKSLSLSPG |
| | (SEQ ID NO: 36) |
| heavy | ASTKGPSVFPLAPCSRSTSESTAALGCLVKDYFPEPVTVSWNSGALT |
| chain | SGVHTFPAVLQSSGLYSLSSVVTVPSSSLGTKTYTCNVDHKPSNTKV |
| constant | DKRVESKYGPPCPPCPAPEFLGGPSVFLFPPKPKDTLMISRTPEVTC |
| region | VVVDVSQEDPEVQFNWYVDGVEVHNAKTKPREEQFNSTYRVVSVLTV |
| (IgG4) | LHQDWLNGKEYKCKVSNKGLPSSIEKTISKAKGQPREPQVYTLPPSQ |
| | EEMTKNQVSLWCLVKGFYPSDIAVEWESNGQPENNYKTTPPVLDSDG |
| | SFFLYSRLTVDKSRWQEGNVFSCSVMHEALHNHYTQKSLSLSLG |
| | (SEQ ID NO: 37) |
| heavy | ASTKGPSVFPLAPSSKSTSGGTAALGCLVKDYFPEPVTVSWNSGALT |
| chain | SGVHTFPAVLQSSGLYSLSSVVTVPSSSLGTQTYICNVNHKPSNTKV |
| constant | DKRVEPKSCDKTHTCPPCPAPEAAGGPSVFLFPPKPKDTLMISRTPE |
| region | VTCVVVDVSHEDPEVKFNWYVDGVEVHNAKTKPREEQYNSTYRVVSV |
| (IgG1) | LTVLHQDWLNGKEYKCKVSNKALPAPIEKTISKAKGQPREPQVYTLP |
| | PSREEMTKNQVSLSCAVKGFYPSDIAVEWESNGQPENNYKTIPPVLD |
| | SDGSFFLVSKLTVDKSRWQQGNVFSCSVMHEALHNRFTQKSLSLSPG |
| | (SEQ ID NO: 38) |
| heavy | AASTKGPSVFPLAPCSRSTSESTAALGCLVKDYFPEPVTVSWNSGAL |
| chain | TSGVHTFPAVLQSSGLYSLSSVVTVPSSSLGTKTYTCNVDHKPSNTK |
| constant | VDKRVESKYGPPCPPCPAPEFLGGPSVFLFPPKPKDTLMISRTPEVT |
| region | CVVVDVSQEDPEVQFNWYVDGVEVHNAKTKPREEQFNSTYRVVSVLT |
| (IgG4) | VLHQDWLNGKEYKCKVSNKGLPSSIEKTISKAKGQPREPQVYTLPPS |
| | QEEMTKNQVSLSCAVKGFYPSDIAVEWESNGQPENNYKTIPPVLDSD |
| | GSFFLVSRLTVDKSRWQEGNVFSCSVMHEALHNRFTQKSLSLSLG |
| | (SEQ ID NO: 39) |

TABLE 2
Linker Sequences

| Linker | |
|---|---|
| length | |
| (number | |
| of amino | |
| acids) | Linker amino acid sequence composition |
| 22 | GGGGSGGGGSGGSGGSGGGGGS |
| | (SEQ ID NO: 6) |
| 26 | GGGGSGGGGSGGGGSGGGGSGGGGGS |
| | (SEQ ID NO: 7) |
| 30 | GGGGSGGGGGGSGGGGGGSGGGGSGGGGGS |
| | (SEQ ID NO: 8) |
| 34 | GGGGSGGGGSGGGSGGGSGGGGSGGGGSGGGGGS |
| | (SEQ ID NO: 9) |
| 38 | GGGGSGGGGSGGGSGGGSGGGSGGGGSGGGGSGGGGGS |
| | (SEQ ID NO: 10) |
| 42 | GGGGSGGGGSGGGSGGGSGGGSGGGGSGGGGGSGGGSG |
| | GGGS (SEQ ID NO: 11) |
| 34 | GGSEGKSSGSGSESKSTEGKSSGSGSESKSTGGS |
| | (SEQ ID NO: 12) |
| 38 | GGSEGKSTSGSGSEGSKSTEGSKSSGSGSESKGSTGGS |
| | (SEQ ID NO: 13) |
| 42 | GGSEGKSTSGSGSEGSKSTEGSKSEGKSTGSGSESKGS |
| | TGGS (SEQ ID NO: 14) |
| 38 | GGGGSEGKSSGSGSESKSTEGKSSGSGSESKSTGGGGS |
| | (SEQ ID NO: 40) |

TABLE 3
Heavy and Light Chain Variable Sequences

| | |
|---|---|
| (SEQ ID | >Adalimumab_light_chain |
| NO: 15) | DIQMTQSPSSLSASVGDRVTITCRASQGIRNYLAWYQQKPGKAPKLL |
| | IYAASTLQSGVPSRFSGSGSGTDFTLTISSLQPEDVATYYCQRYNRA |
| | PYTFGQGTKVEIK |
| (SEQ ID | >Adalimumab_heavy_chain |
| NO: 16) | EVQLVESGGGLVQPGRSLRLSCAASGFTFDDYAMHWVRQAPGKGLEW |
| | VSAITWNSGHIDYADSVEGRFTISRDNAKNSLYLQMNSLRAEDTAVY |
| | YCAKVSYLSTASSLDYWGQGTLVTVSS |
| (SEQ ID | >Certolizumab _light_chain |
| NO: 17) | DIQMTQSPSSLSASVGDRVTITCKASQNVGTNVAWYQQKPGKAPKAL |
| | IYSASFLYSGVPYRFSGSGSGTDFTLTISSLQPEDFATYYCQQYNIY |
| | PLTFGQGTKVEIK |
| (SEQ ID | >Certolizumab _heavy_chain |
| NO: 18) | EVQLVESGGGLVQPGGSLRLSCAASGYVFTDYGMNWVRQAPGKGLEW |
| | MGWINTYIGEPIYADSVKGRFTFSLDTSKSTAYLQMNSLRAEDTAVY |
| | YCARGYRSYAMDYWGQGTLVTVSS |
| (SEQ ID | >Ustekinumab_light_chain |
| NO: 19) | DIQMTQSPSSLSASVGDRVTITCRASQGISSWLAWYQQKPEKAPKSL |
| | IYAASSLQSGVPSRFSGSGSGTDFTLTISSLQPEDFATYYCQQYNIY |
| | PYTFGQGTKLEIK |
| (SEQ ID | >Ustekinumab_heavy_chain |
| NO: 20) | EVQLVQSGAEVKKPGESLKISCKGSGYSFTTYWLGWVRQMPGKGLDW |
| | IGIMSPVDSDIRYSPSFQGQVTMSVDKSITTAYLQWNSLKASDTAMY |
| | YCARRRPGQGYFDFWGQGTLVTVSS |
| (SEQ ID | >Ixekizumab_light_chain |
| NO: 21) | DIVMTQTPLSLSVTPGQPASISCRSSRSLVHSRGNTYLHWYLQKPGQ |
| | SPQLLIYKVSNRFIGVPDRFSGSGSGTDFTLKISRVEAEDVGVYYCS |
| | QSTHLPFTFGQGTKLEIK |
| (SEQ ID | >Ixekizumab_heavy_chain |
| NO: 22) | QVQLVQSGAEVKKPGSSVKVSCKASGYSFTDYHIHWVRQAPGQGLEW |
| | MGVINPMYGTTDYNQRFKGRVTITADESTSTAYMELSSLRSEDTAVY |
| | YCARYDYFTGTGVYWGQGTLVTVSS |
| (SEQ ID | >Epcam_light_chain |
| NO: 41) | ELVMTQSPSSLTVTAGEKVTMSCKSSQSLLNSGNQKNYLTWYQQKPG |
| | QPPKLLIYWASTRESGVPDRFTGSGSGTDFTLTISSVQAEDLAVYYC |
| | QNDYSYPLTFGAGTKLEIK |
| (SEQ ID | >Epcam_heavy_chain |
| NO: 42) | EVQLLEQSGAELVRPGTSVKISCKASGYAFTNYWLGWVKQRPGHGLE |
| | WIGDIFPGSGNIHYNEKFKGKATLTADKSSSTAYMQLSSLTFEDSAV |
| | YFCARLRNWDEPMDYWGQGTTVTVSS |
| (SEQ ID | >FAP_light_chain |
| NO: 43) | QIVLTQSPAIMSASPGEKVTMTCSASSGVNFMHWYQQKSGTSPKRWI |
| | FDTSKLASGVPARFSGSGSGTSYSLTISSMEAEDAATYYCQQWSFNP |
| | PTFGGGTKLEIK |
| (SEQ ID | >FAP_heavy_chain |
| NO: 44) | QVQLQQSGAELARPGASVNLSCKASGYTFTNNGINWLKQRTGQGLEW |
| | IGEIYPRSTNTLYNEKFKGKATLTADRSSNTAYMELRSLTSEDSAVY |
| | FCARTLTAPFAFWGQGTLVTVSA |
| (SEQ ID | > Lebrikizumab light_chain |
| NO: 45) | DIVMTQSPDSLSVSLGERATINCRASKSVDSYGNSFMHWYQQKPGQP |
| | PKLLIYLASNLESGVPDRFSGSGSGTDFTLTISSLQAEDVAVYYCQQ |
| | NNEDPRTFGGGTKVEIK |
| (SEQ ID | > Lebrikizumab heavy_chain |
| NO: 46) | QVTLRESGPALVKPTQTLTLTCTVSGFSLSAYSVNWIRQPPGKALEW |
| | LAMIWGDGKIVYNSALKSRLTISKDTSKNQVVLTMTNMDPVDTATYY |
| | CAGDGYYPYAMDNWGQGSLVTVSS |
| (SEQ ID | > CD33_light_chain |
| NO: 47) | DIVMTQSPDSLTVSLGERTTINCKSSQSVLDSSKNKNSLAWYQQKPG |
| | QPPKLLLSWASTRESGIPDRFSGSGSGTDFTLTIDSLQPEDSATYYC |
| | QQSAHFPITFGQGTRLEIK |
| (SEQ ID | > CD33_heavy_chain |
| NO: 48) | QVQLVQSGAEVKKPGASVKVSCKASGYTFTNYGMNWVKQAPGQGLKW |
| | MGWINTYTGEPTYADDFKGRVTMTSDTSTSTAYLELHNLRSDDTAVY |
| | YCARWSWSDGYYVYFDYWGQGTTVTVSS |

TABLE 4
Heavy chains and light chains; chains comprising a light and a heavy chain

| | |
|---|---|
| heavy chain | EVQLVESGGGLVQPGRSLRLSCAASGFTFDDYAMHWVRQAPGKG |
| (SEQ ID NOs: 1 | LEWVSAITWNSGHIDYADSVEGRFTISRDNAKNSLYLQMNSLRA |
| and 16) | EDTAVYYCAKVSYLSTASSLDYWGQGTLVTVSSASTKGPSVFPL |
| | APSSKSTSGGTAALGCLVKDYFPEPVTVSWNSGALTSGVHTFPA |
| | VLQSSGLYSLSSVVTVPSSSLGTQTYICNVNHKPSNTKVDKRVE |
| | PKSCDKTHTCPPCPAPELLGGPSVFLFPPKPKDTLMISRTPEVT |
| | CVVVDVSHEDPEVKFNWYVDGVEVHNAKTKPREEQYNSTYRVVS |
| | VLTVLHQDWLNGKEYKCKVSNKALPAPIEKTISKAKGQPREPQV |
| | YTLPPSREEMTKNQVSLYCLVKGFYPSDIAVEWESNGQPENNYK |
| | TTPPVLDSDGSFFLYSKLTVDKSRWQQGNVFSCSVMHEALHNHY |
| | TQKSLSLSPGK (SEQ ID NO: 23) |
| light chain | DIQMTQSPSSLSASVGDRVTITCRASQGIRNYLAWYQQKPGKAP |
| (SEQ ID NOs: 2 | KLLIYAASTLQSGVPSRFSGSGSGTDFTLTISSLQPEDVATYYC |
| and 15) | QRYNRAPYTFGQGTKVEIKRTVAAPSVFIFPPSDEQLKSGTASV |
| | VCLLNNFYPREAKVQWKVDNALQSGNSQESVTEQDSKDSTYSLS |
| | STLTLSKADYEKHKVYACEVTHQGLSSPVTKSFNRGEC (SEQ |
| | ID NO: 24) |
| heavy chain | EVQLVESGGGLVQPGGSLRLSCAASGYVFTDYGMNWVRQAPGKG |
| (SEQ ID NOs: 3 | LEWMGWINTYIGEPIYADSVKGRFTFSLDTSKSTAYLQMNSLRA |
| and 18) | EDTAVYYCARGYRSYAMDYWGQGTLVTVSSASTKGPSVFPLAPS |
| | SKSTSGGTAALGCLVKDYFPEPVTVSWNSGALTSGVHTFPAVLQ |
| | SSGLYSLSSVVTVPSSSLGTQTYICNVNHKPSNTKVDKRVEPKS |
| | CDKTHTCPPCPAPELLGGPSVFLFPPKPKDTLMISRTPEVTCVV |
| | VDVSHEDPEVKFNWYVDGVEVHNAKTKPREEQYNSTYRVVSVLT |
| | VLHQDWLNGKEYKCKVSNKALPAPIEKTISKAKGQPREPQVYTL |
| | PPSREEMTKNQVSLTCLVKGFYPSDIAVEWESNGQPENNYKTTP |
| | PVLDSDGSFFLTSKLTVDKSRWQQGNVFSCSVMHEALHNRFTQK |
| | SLSLSPGK (SEQ ID NO: 25) |
| light chain | DIQMTQSPSSLSASVGDRVTITCKASQNVGTNVAWYQQKPGKAP |
| (SEQ ID NOs: 2 | KALIYSASFLYSGVPYRFSGSGSGTDFTLTISSLQPEDFATYYC |
| and 17) | QQYNIYPLTFGQGTKVEIKRTVAAPSVFIFPPSDEQLKSGTASV |
| | VCLLNNFYPREAKVQWKVDNALQSGNSQESVTEQDSKDSTYSLS |
| | STLTLSKADYEKHKVYACEVTHQGLSSPVTKSFNRGEC (SEQ |
| | ID NO: 26) |
| heavy chain | EVQLVQSGAEVKKPGESLKISCKGSGYSFTTYWLGWVRQMPGKG |
| (SEQ ID NOs: 1 | LDWIGIMSPVDSDIRYSPSFQGQVTMSVDKSITTAYLQWNSLKA |
| and 20) | SDTAMYYCARRRPGQGYFDFWGQGTLVTVSSASTKGPSVFPLAP |
| | SSKSTSGGTAALGCLVKDYFPEPVTVSWNSGALTSGVHTFPAVL |
| | QSSGLYSLSSVVTVPSSSLGTQTYICNVNHKPSNTKVDKRVEPK |
| | SCDKTHTCPPCPAPELLGGPSVFLFPPKPKDTLMISRTPEVTCV |
| | VVDVSHEDPEVKFNWYVDGVEVHNAKTKPREEQYNSTYRVVSVL |
| | TVLHQDWLNGKEYKCKVSNKALPAPIEKTISKAKGQPREPQVYT |
| | LPPSREEMTKNQVSLYCLVKGFYPSDIAVEWESNGQPENNYKTT |
| | PPVLDSDGSFFLYSKLTVDKSRWQQGNVFSCSVMHEALHNHYTQ |
| | KSLSLSPGK (SEQ ID NO: 27) |
| light chain | DIQMTQSPSSLSASVGDRVTITCRASQGISSWLAWYQQKPEKAP |
| (SEQ ID NOs: 2 | KSLIYAASSLQSGVPSRFSGSGSGTDFTLTISSLQPEDFATYYC |
| and 19) | QQYNIYPYTFGQGTKLEIKRTVAAPSVFIFPPSDEQLKSGTASV |
| | VCLLNNFYPREAKVQWKVDNALQSGNSQESVTEQDSKDSTYSLS |
| | STLTLSKADYEKHKVYACEVTHQGLSSPVTKSFNRGEC (SEQ |
| | ID NO: 28) |
| heavy chain | QVQLVQSGAEVKKPGSSVKVSCKASGYSFTDYHIHWVRQAPGQG |
| (SEQ ID NOs: 3 | LEWMGVINPMYGTTDYNQRFKGRVTITADESTSTAYMELSSLRS |
| and 22) | EDTAVYYCARYDYFTGTGVYWGQGTLVTVSSASTKGPSVFPLAP |
| | SSKSTSGGTAALGCLVKDYFPEPVTVSWNSGALTSGVHTFPAVL |
| | QSSGLYSLSSVVTVPSSSLGTQTYICNVNHKPSNTKVDKRVEPK |
| | SCDKTHTCPPCPAPELLGGPSVFLFPPKPKDTLMISRTPEVTCV |
| | VVDVSHEDPEVKFNWYVDGVEVHNAKTKPREEQYNSTYRVVSVL |
| | TVLHQDWLNGKEYKCKVSNKALPAPIEKTISKAKGQPREPQVYT |
| | LPPSREEMTKNQVSLTCLVKGFYPSDIAVEWESNGQPENNYKTT |
| | PPVLDSDGSFFLTSKLTVDKSRWQQGNVFSCSVMHEALHNRFTQ |
| | KSLSLSPGK (SEQ ID NO: 29) |
| light chain | DIVMTQTPLSLSVTPGQPASISCRSSRSLVHSRGNTYLHWYLQK |
| (SEQ ID NOs: 2 | PGQSPQLLIYKVSNRFIGVPDRFSGSGSGTDFTLKISRVEAEDV |
| and 21) | GVYYCSQSTHLPFTFGQGTKLEIKRTVAAPSVFIFPPSDEQLKS |
| | GTASVVCLLNNFYPREAKVQWKVDNALQSGNSQESVTEQDSKDS |
| | TYSLSSTLTLSKADYEKHKVYACEVTHQGLSSPVTKSFNRGEC |
| | (SEQ ID NO: 30) |
| heavy chain | EVQLVESGGGLVQPGRSLRLSCAASGFTFDDYAMHWVRQAPGKG |
| (SEQ ID NOs: 4 | LEWVSAITWNSGHIDYADSVEGRFTISRDNAKNSLYLQMNSLRA |
| and 16) | EDTAVYYCAKVSYLSTASSLDYWGQGTLVTVSSASTKGPSVFPL |
| | APSSKSTSGGTAALGCLVKDYFPEPVTVSWNSGALTSGVHTFPA |
| | VLQSSGLYSLSSVVTVPSSSLGTQTYICNVNHKPSNTKVDKRVE |
| | PKSCDKTHTCPPCPAPELLGGPSVFLFPPKPKDTLYITREPEVT |
| | CVVVDVSHEDPEVKFNWYVDGVEVHNAKTKPREEQYNSTYRVVS |
| | VLTVLHQDWLNGKEYKCKVSNKALPAPIEKTISKAKGQPREPQV |
| | YTLPPSREEMTKNQVSLYCLVKGFYPSDIAVEWESNGQPENNYK |
| | TTPPVLDSDGSFFLYSKLTVDKSRWQQGNVFSCSVMHEALHNHY |
| | TQKSLSLSPGK (SEQ ID NO: 31) |
| heavy chain | EVQLVESGGGLVQPGGSLRLSCAASGYVFTDYGMNWVRQAPGKG |
| (SEQ ID NOS: 5 | LEWMGWINTYIGEPIYADSVKGRFTFSLDTSKSTAYLQMNSLRA |
| and 18) | EDTAVYYCARGYRSYAMDYWGQGTLVTVSSASTKGPSVFPLAPS |
| | SKSTSGGTAALGCLVKDYFPEPVTVSWNSGALTSGVHTFPAVLQ |
| | SSGLYSLSSVVTVPSSSLGTQTYICNVNHKPSNTKVDKRVEPKS |
| | CDKTHTCPPCPAPELLGGPSVFLFPPKPKDTLYITREPEVTCVV |
| | VDVSHEDPEVKFNWYVDGVEVHNAKTKPREEQYNSTYRVVSVLT |
| | VLHQDWLNGKEYKCKVSNKALPAPIEKTISKAKGQPREPQVYTL |
| | PPSREEMTKNQVSLTCLVKGFYPSDIAVEWESNGQPENNYKTTP |
| | PVLDSDGSFFLTSKLTVDKSRWQQGNVFSCSVMHEALHNRFTQK |
| | SLSLSPGK (SEQ ID NO: 32) |
| heavy chain | EVQLVQSGAEVKKPGESLKISCKGSGYSFTTYWLGWVRQMPGKG |
| (SEQ ID NOs: 4 | LDWIGIMSPVDSDIRYSPSFQGQVTMSVDKSITTAYLQWNSLKA |
| and 20) | SDTAMYYCARRRPGQGYFDFWGQGTLVTVSSASTKGPSVFPLAP |
| | SSKSTSGGTAALGCLVKDYFPEPVTVSWNSGALTSGVHTFPAVL |
| | QSSGLYSLSSVVTVPSSSLGTQTYICNVNHKPSNTKVDKRVEPK |
| | SCDKTHTCPPCPAPELLGGPSVFLFPPKPKDTLYITREPEVTCV |
| | VVDVSHEDPEVKFNWYVDGVEVHNAKTKPREEQYNSTYRVVSVL |
| | TVLHQDWLNGKEYKCKVSNKALPAPIEKTISKAKGQPREPQVYT |
| | LPPSREEMTKNQVSLYCLVKGFYPSDIAVEWESNGQPENNYKTT |
| | PPVLDSDGSFFLYSKLTVDKSRWQQGNVFSCSVMHEALHNHYTQ |
| | KSLSLSPGK (SEQ ID NO: 33) |
| heavy chain | QVQLVQSGAEVKKPGSSVKVSCKASGYSFTDYHIHWVRQAPGQG |
| (SEQ ID NOs: 5 | LEWMGVINPMYGTTDYNQRFKGRVTITADESTSTAYMELSSLRS |
| and 22) | EDTAVYYCARYDYFTGTGVYWGQGTLVTVSSASTKGPSVFPLAP |
| | SSKSTSGGTAALGCLVKDYFPEPVTVSWNSGALTSGVHTFPAVL |
| | QSSGLYSLSSVVTVPSSSLGTQTYICNVNHKPSNTKVDKRVEPK |
| | SCDKTHTCPPCPAPELLGGPSVFLFPPKPKDTLYITREPEVTCV |
| | VVDVSHEDPEVKFNWYVDGVEVHNAKTKPREEQYNSTYRVVSVL |
| | TVLHQDWLNGKEYKCKVSNKALPAPIEKTISKAKGQPREPQVYT |
| | LPPSREEMTKNQVSLTCLVKGFYPSDIAVEWESNGQPENNYKTT |
| | PPVLDSDGSFFLTSKLTVDKSRWQQGNVFSCSVMHEALHNRFTQ |
| | KSLSLSPGK (SEQ ID NO: 34) |
| chain | ELVMTQSPSSLTVTAGEKVTMSCKSSQSLLNSGNQKNYLTWYQQ |
| comprising a | KPGQPPKLLIYWASTRESGVPDRFTGSGSGTDFTLTISSVQAED |
| light chain | LAVYYCQNDYSYPLTFGAGTKLEIKRTVAAPSVFIFPPSDEQLK |
| and a heavy | SGTASVVCLLNNFYPREAKVQWKVDNALQSGNSQESVTEQDSKD |
| chain (SEQ ID | STYSLSSTLTLSKADYEKHKVYACEVTHQGLSSPVTKSFNRGEC |
| NOs: 41, 2, | GGGGSEGKSSGSGSESKSTEGKSSGSGSESKSTGGGGSEVQLLE |
| 40, 42 and 36) | QSGAELVRPGTSVKISCKASGYAFTNYWLGWVKQRPGHGLEWIG |
| | DIFPGSGNIHYNEKFKGKATLTADKSSSTAYMQLSSLTFEDSAV |
| | YFCARLRNWDEPMDYWGQGTTVTVSSASTKGPSVFPLAPSSKST |
| | SGGTAALGCLVKDYFPEPVTVSWNSGALTSGVHTFPAVLQSSGL |
| | YSLSSVVTVPSSSLGTQTYICNVNHKPSNTKVDKRVEPKSCDKT |
| | HTCPPCPAPEAAGGPSVFLFPPKPKDTLMISRTPEVTCVVVDVS |
| | HEDPEVKFNWYVDGVEVHNAKTKPREEQYNSTYRVVSVLTVLHQ |
| | DWLNGKEYKCKVSNKALPAPIEKTISKAKGQPREPQVYTLPPSR |
| | EEMTKNQVSLWCLVKGFYPSDIAVEWESNGQPENNYKTTPPVLD |
| | SDGSFFLYSKLTVDKSRWQQGNVFSCSVMHEALHNHYTQKSLSL |
| | SPG (SEQ ID NO: 49) |
| chain | ELVMTQSPSSLTVTAGEKVTMSCKSSQSLLNSGNQKNYLTWYQQ |
| comprising a | KPGQPPKLLIYWASTRESGVPDRFTGSGSGTDFTLTISSVQAED |
| light chain | LAVYYCQNDYSYPLTFGAGTKLEIKRTVAAPSVFIFPPSDEQLK |
| and a heavy | SGTASVVCLLNNFYPREAKVQWKVDNALQSGNSQESVTEQDSKD |
| chain (SEQ ID | STYSLSSTLTLSKADYEKHKVYACEVTHQGLSSPVTKSFNRGEC |
| NOs: 41, 2, | GGGGSEGKSSGSGSESKSTEGKSSGSGSESKSTGGGGSEVQLLE |
| 40, 42 and 37) | QSGAELVRPGTSVKISCKASGYAFTNYWLGWVKQRPGHGLEWIG |
| | DIFPGSGNIHYNEKFKGKATLTADKSSSTAYMQLSSLTFEDSAV |
| | YFCARLRNWDEPMDYWGQGTTVTVSSASTKGPSVFPLAPCSRST |
| | SESTAALGCLVKDYFPEPVTVSWNSGALTSGVHTFPAVLQSSGL |
| | YSLSSVVTVPSSSLGTKTYTCNVDHKPSNTKVDKRVESKYGPPC |
| | PPCPAPEFLGGPSVFLFPPKPKDTLMISRTPEVTCVVVDVSQED |
| | PEVQFNWYVDGVEVHNAKTKPREEQFNSTYRVVSVLTVLHQDWL |
| | NGKEYKCKVSNKGLPSSIEKTISKAKGQPREPQVYTLPPSQEEM |
| | TKNQVSLWCLVKGFYPSDIAVEWESNGQPENNYKTTPPVLDSDG |
| | SFFLYSRLTVDKSRWQEGNVFSCSVMHEALHNHYTQKSLSLSLG |
| | (SEQ ID NO: 50) |
| chain | DIVMTQSPDSLTVSLGERTTINCKSSQSVLDSSKNKNSLAWYQQ |
| comprising a | KPGQPPKLLLSWASTRESGIPDRFSGSGSGTDFTLTIDSLQPED |
| light chain | SATYYCQQSAHFPITFGQGTRLEIKRTVAAPSVFIFPPSDEQLK |
| and a heavy | SGTASVVCLLNNFYPREAKVQWKVDNALQSGNSQESVTEQDSKD |
| chain (SEQ ID | STYSLSSTLTLSKADYEKHKVYACEVTHQGLSSPVTKSFNRGEC |
| NOs: 47, 2, | GGGGSEGKSSGSGSESKSTEGKSSGSGSESKSTGGGGSQVQLVQ |
| 40, 48 and 38) | SGAEVKKPGASVKVSCKASGYTFTNYGMNWVKQAPGQGLKWMGW |
| | INTYTGEPTYADDFKGRVTMTSDTSTSTAYLELHNLRSDDTAVY |
| | YCARWSWSDGYYVYFDYWGQGTTVTVSSASTKGPSVFPLAPSSK |
| | STSGGTAALGCLVKDYFPEPVTVSWNSGALTSGVHTFPAVLQSS |
| | GLYSLSSVVTVPSSSLGTQTYICNVNHKPSNTKVDKRVEPKSCD |
| | KTHTCPPCPAPEAAGGPSVFLFPPKPKDTLMISRTPEVTCVVVD |
| | VSHEDPEVKFNWYVDGVEVHNAKTKPREEQYNSTYRVVSVLTVL |
| | HQDWLNGKEYKCKVSNKALPAPIEKTISKAKGQPREPQVYTLPP |
| | SREEMTKNQVSLSCAVKGFYPSDIAVEWESNGQPENNYKTTPPV |
| | LDSDGSFFLVSKLTVDKSRWQQGNVFSCSVMHEALHNRFTQKSL |
| | SLSPG (SEQ ID NO: 51) |
| chain | DIVMTQSPDSLTVSLGERTTINCKSSQSVLDSSKNKNSLAWYQQ |
| comprising a | KPGQPPKLLLSWASTRESGIPDRFSGSGSGTDFTLTIDSLQPED |
| light chain | SATYYCQQSAHFPITFGQGTRLEIKRTVAAPSVFIFPPSDEQLK |
| and a heavy | SGTASVVCLLNNFYPREAKVQWKVDNALQSGNSQESVTEQDSKD |
| chain (SEQ ID | STYSLSSTLTLSKADYEKHKVYACEVTHQGLSSPVTKSFNRGEC |
| NOs: 47, 2, | GGGGSEGKSSGSGSESKSTEGKSSGSGSESKSTGGGGSQVQLVQ |
| 40, 48 and 39) | SGAEVKKPGASVKVSCKASGYTFTNYGMNWVKQAPGQGLKWMGW |
| | INTYTGEPTYADDFKGRVTMTSDTSTSTAYLELHNLRSDDTAVY |
| | YCARWSWSDGYYVYFDYWGQGTTVTVSSASTKGPSVFPLAPCSR |
| | STSESTAALGCLVKDYFPEPVTVSWNSGALTSGVHTFPAVLQSS |
| | GLYSLSSVVTVPSSSLGTKTYTCNVDHKPSNTKVDKRVESKYGP |
| | PCPPCPAPEFLGGPSVFLFPPKPKDTLMISRTPEVTCVVVDVSQ |
| | EDPEVQFNWYVDGVEVHNAKTKPREEQFNSTYRVVSVLTVLHQD |
| | WLNGKEYKCKVSNKGLPSSIEKTISKAKGQPREPQVYTLPPSQE |
| | EMTKNQVSLSCAVKGFYPSDIAVEWESNGQPENNYKTTPPVLDS |
| | DGSFFLVSRLTVDKSRWQEGNVFSCSVMHEALHNRFTQKSLSLS |
| | LG (SEQ ID NO: 52) |
| chain | QIVLTQSPAIMSASPGEKVTMTCSASSGVNFMHWYQQKSGTSPK |
| comprising a | RWIFDTSKLASGVPARFSGSGSGTSYSLTISSMEAEDAATYYCQ |
| light chain | QWSFNPPTFGGGTKLEIKRTVAAPSVFIFPPSDEQLKSGTASVV |
| and a heavy | CLLNNFYPREAKVQWKVDNALQSGNSQESVTEQDSKDSTYSLSS |
| chain (SEQ ID | TLTLSKADYEKHKVYACEVTHQGLSSPVTKSFNRGECGGGGSEG |
| NOs: 43, 2, | KSSGSGSESKSTEGKSSGSGSESKSTGGGGSQVQLQQSGAELAR |
| 40, 44 and 36) | PGASVNLSCKASGYTFTNNGINWLKQRTGQGLEWIGEIYPRSTN |
| | TLYNEKFKGKATLTADRSSNTAYMELRSLTSEDSAVYFCARTLT |
| | APFAFWGQGTLVTVSAASTKGPSVFPLAPSSKSTSGGTAALGCL |
| | VKDYFPEPVTVSWNSGALTSGVHTFPAVLQSSGLYSLSSVVTVP |
| | SSSLGTQTYICNVNHKPSNTKVDKRVEPKSCDKTHTCPPCPAPE |
| | AAGGPSVFLFPPKPKDTLMISRTPEVTCVVVDVSHEDPEVKFNW |
| | YVDGVEVHNAKTKPREEQYNSTYRVVSVLTVLHQDWLNGKEYKC |
| | KVSNKALPAPIEKTISKAKGQPREPQVYTLPPSREEMTKNQVSL |
| | WCLVKGFYPSDIAVEWESNGQPENNYKTTPPVLDSDGSFFLYSK |
| | LTVDKSRWQQGNVFSCSVMHEALHNHYTQKSLSLSPG (SEQ |
| | ID NO: 53) |
| chain | QIVLTQSPAIMSASPGEKVTMTCSASSGVNFMHWYQQKSGTSPK |
| comprising a | RWIFDTSKLASGVPARFSGSGSGTSYSLTISSMEAEDAATYYCQ |
| light chain | QWSFNPPTFGGGTKLEIKRTVAAPSVFIFPPSDEQLKSGTASVV |
| and a heavy | CLLNNFYPREAKVQWKVDNALQSGNSQESVTEQDSKDSTYSLSS |
| chain (SEQ ID | TLTLSKADYEKHKVYACEVTHQGLSSPVTKSFNRGECGGGGSEG |
| NOs: 43, 2, | KSSGSGSESKSTEGKSSGSGSESKSTGGGGSQVQLQQSGAELAR |
| 40, 44 and 37) | PGASVNLSCKASGYTFTNNGINWLKQRTGQGLEWIGEIYPRSTN |
| | TLYNEKFKGKATLTADRSSNTAYMELRSLTSEDSAVYFCARTLT |
| | APFAFWGQGTLVTVSAASTKGPSVFPLAPCSRSTSESTAALGCL |
| | VKDYFPEPVTVSWNSGALTSGVHTFPAVLQSSGLYSLSSVVTVP |
| | SSSLGTKTYTCNVDHKPSNTKVDKRVESKYGPPCPPCPAPEFLG |
| | GPSVFLFPPKPKDTLMISRTPEVTCVVVDVSQEDPEVQFNWYVD |
| | GVEVHNAKTKPREEQFNSTYRVVSVLTVLHQDWLNGKEYKCKVS |
| | NKGLPSSIEKTISKAKGQPREPQVYTLPPSQEEMTKNQVSLWCL |
| | VKGFYPSDIAVEWESNGQPENNYKTTPPVLDSDGSFFLYSRLTV |
| | DKSRWQEGNVFSCSVMHEALHNHYTQKSLSLSLG (SEQ ID |
| | NO: 54) |
| chain | DIVMTQSPDSLSVSLGERATINCRASKSVDSYGNSFMHWYQQKP |
| comprising a | GQPPKLLIYLASNLESGVPDRFSGSGSGTDFTLTISSLQAEDVA |
| light chain | VYYCQQNNEDPRTFGGGTKVEIKRTVAAPSVFIFPPSDEQLKSG |
| and a heavy | TASVVCLLNNFYPREAKVQWKVDNALQSGNSQESVTEQDSKDST |
| chain (SEQ ID | YSLSSTLTLSKADYEKHKVYACEVTHQGLSSPVTKSFNRGECGG |
| NOs: 45, 2, | GGSEGKSSGSGSESKSTEGKSSGSGSESKSTGGGGSQVTLRESG |
| 40, 46 and 36) | PALVKPTQTLTLTCTVSGFSLSAYSVNWIRQPPGKALEWLAMIW |
| | GDGKIVYNSALKSRLTISKDTSKNQVVLTMTNMDPVDTATYYCA |
| | GDGYYPYAMDNWGQGSLVTVSSASTKGPSVFPLAPSSKSTSGGT |
| | AALGCLVKDYFPEPVTVSWNSGALTSGVHTFPAVLQSSGLYSLS |
| | SVVTVPSSSLGTQTYICNVNHKPSNTKVDKRVEPKSCDKTHTCP |
| | PCPAPEAAGGPSVFLFPPKPKDTLMISRTPEVTCVVVDVSHEDP |
| | EVKFNWYVDGVEVHNAKTKPREEQYNSTYRVVSVLTVLHQDWLN |
| | GKEYKCKVSNKALPAPIEKTISKAKGQPREPQVYTLPPSREEMT |
| | KNQVSLWCLVKGFYPSDIAVEWESNGQPENNYKTTPPVLDSDGS |
| | FFLYSKLTVDKSRWQQGNVFSCSVMHEALHNHYTQKSLSLSPG |
| | (SEQ ID NO: 55) |
| chain | DIVMTQSPDSLSVSLGERATINCRASKSVDSYGNSFMHWYQQKP |
| comprising a | GQPPKLLIYLASNLESGVPDRFSGSGSGTDFTLTISSLQAEDVA |
| light chain | VYYCQQNNEDPRTFGGGTKVEIKRTVAAPSVFIFPPSDEQLKSG |
| and a heavy | TASVVCLLNNFYPREAKVQWKVDNALQSGNSQESVTEQDSKDST |
| chain (SEQ ID | YSLSSTLTLSKADYEKHKVYACEVTHQGLSSPVTKSFNRGECGG |
| NOs: 45, 2, | GGSEGKSSGSGSESKSTEGKSSGSGSESKSTGGGGSQVTLRESG |
| 40, 46 and 37) | PALVKPTQTLTLTCTVSGFSLSAYSVNWIRQPPGKALEWLAMIW |
| | GDGKIVYNSALKSRLTISKDTSKNQVVLTMTNMDPVDTATYYCA |
| | GDGYYPYAMDNWGQGSLVTVSSASTKGPSVFPLAPCSRSTSEST |
| | AALGCLVKDYFPEPVTVSWNSGALTSGVHTFPAVLQSSGLYSLS |
| | SVVTVPSSSLGTKTYTCNVDHKPSNTKVDKRVESKYGPPCPPCP |
| | APEFLGGPSVFLFPPKPKDTLMISRTPEVTCVVVDVSQEDPEVQ |
| | FNWYVDGVEVHNAKTKPREEQFNSTYRVVSVLTVLHQDWLNGKE |
| | YKCKVSNKGLPSSIEKTISKAKGQPREPQVYTLPPSQEEMTKNQ |
| | VSLWCLVKGFYPSDIAVEWESNGQPENNYKTTPPVLDSDGSFFL |
| | YSRLTVDKSRWQEGNVFSCSVMHEALHNHYTQKSLSLSLG |
| | (SEQ ID NO: 56) |

### Antibody Modifications

Antibodies can include modifications. For example, it may be desirable to modify the antibody with respect to effector function, so as to enhance the effectiveness of the antibody in treating cancer. One such modification is the introduction of cysteine residue(s) into the Fc region, thereby allowing interchain disulfide bond formation in this region. The homodimeric antibody thus generated can have improved internalization capability and/or increased complement-mediated cell killing and/or antibody-dependent cellular cytotoxicity (ADCC). See, for example, Caron et al., 1992, J. Exp Med. 176:1191-1195; and Shopes, 1992, J. Immunol. 148:2918-2922. Homodimeric antibodies having enhanced anti-tumor activity can also be prepared using heterobifunctional cross-linkers as described in Wolff et al., 1993, Cancer Research 53: 2560-2565. Alternatively, an antibody can be engineered to contain dual Fc regions, enhancing complement lysis and ADCC capabilities of the antibody. See Stevenson et al., 1989, Anti-Cancer Drug Design 3: 219-230.

Antibodies with improved ability to support ADCC have been generated by modifying the glycosylation pattern of their Fc region. This is possible since antibody glycosylation at the asparagine residue, N297, in the C_{H2} domain is involved in the interaction between IgG and Fcγ receptors prerequisite to ADCC. Host cell lines have been engineered to express antibodies with altered glycosylation, such as increased bisecting N-acetylglucosamine or reduced fucose. Fucose reduction provides greater enhancement to ADCC activity than does increasing the presence of bisecting N-acetylglucosamine. Moreover, enhancement of ADCC by low fucose antibodies is independent of the FcγRIIIa V/F polymorphism.

Modifying the amino acid sequence of the Fc region of antibodies is an alternative to glycosylation engineering to enhance ADCC. The binding site on human IgG₁ for Fcγ receptors has been determined by extensive mutational analysis. This led to the generation of IgG₁ antibodies with Fc mutations that increase the binding affinity for FcγRIIIa and enhance ADCC in vitro. Additionally, Fc variants have been obtained with many different permutations of binding properties, e.g., improved binding to specific FcγR receptors with unchanged or diminished binding to other FcγR receptors.

Another aspect includes immunoconjugates comprising the antibody or fragments thereof conjugated to a cytotoxic agent such as a chemotherapeutic agent, a toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used to form useful immunoconjugates include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, the tricothecenes, and the like. A variety of radionuclides are available for the production of radioconjugated antibodies. Examples include ²¹²Bi, ¹³¹I, ¹³¹In, ⁹⁰Y, and ¹⁸⁶Re.

Conjugates of an antibody and cytotoxic or chemotherapeutic agent can be made by known methods, using a variety of bifunctional protein coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., 1987, Science 238:1098. Carbon-14-labeled 1-isothiocyanatobenzyl-3-methyldiethylene triaminepentaacetic acid (MX-DTPA) is an exemplary chelating agent for conjugation of radionucleotide to the antibody. Conjugates also can be formed with a cleavable linker.

Antibodies disclosed herein can also be formulated as immunoliposomes. Liposomes containing the antibody are prepared by methods known in the art, such as described in Epstein et al., 1985, Proc. Natl. Acad. Sci. USA 82:3688; Hwang et al., 1980, Proc. Natl. Acad. Sci. USA 77:4030; and U.S. Pat. Nos. 4,485,045 and 4,544,545. Liposomes having enhanced circulation time are disclosed, for example, in U.S. Pat. No. 5,013,556.

Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of an antibody disclosed herein can be conjugated to the liposomes as described in Martin et al., 1982, J. Biol. Chem. 257:286-288 via a disulfide interchange reaction. A chemotherapeutic agent (such as doxorubicin) is optionally contained within the liposome. See, e.g., Gabizon et al., 1989, J. National Cancer Inst. 81(19):1484.

The antibodies described and disclosed herein can also be used in ADEPT (Antibody-Directed Enzyme Prodrug Therapy) procedures by conjugating the antibody to a prodrug-activating enzyme that converts a prodrug (e.g., a peptidyl chemotherapeutic agent), to an active anti-cancer drug. See, for example, WO 81/01145, WO 88/07378, and U.S. Pat. No. 4,975,278. The enzyme component of the immunoconjugate useful for ADEPT is an enzyme capable of acting on a prodrug in such a way so as to covert it into its more active, cytotoxic form. Specific enzymes that are useful in ADEPT include, but are not limited to, alkaline phosphatase for converting phosphate-containing prodrugs into free drugs; arylsulfatase for converting sulfate-containing prodrugs into free drugs; cytosine deaminase for converting non-toxic 5-fluorocytosine into the anti-cancer drug, 5-fluorouracil; proteases, such as serratia protease, thermolysin, subtilisin, carboxypeptidases, and cathepsins (such as cathepsins B and L), for converting peptide-containing prodrugs into free drugs; D-alanylcarboxypeptidases, for converting prodrugs containing D-amino acid substituents; carbohydrate-cleaving enzymes such as β-galactosidase and neuraminidase for converting glycosylated prodrugs into free drugs; β-lactamase for converting drugs derivatized with β-lactams into free drugs; and penicillin amidases, such as penicillin V amidase or penicillin G amidase, for converting drugs derivatized at their amine nitrogens with phenoxyacetyl or phenylacetyl groups, respectively, into free drugs. Alternatively, antibodies having enzymatic activity (“abzymes”) can be used to convert the prodrugs into free active drugs (see, for example, Massey, 1987, Nature 328: 457-458). Antibody-abzyme conjugates can be prepared by known methods for delivery of the abzyme to a tumor cell population, for example, by covalently binding the enzyme to the antibody/heterobifunctional crosslinking reagents discussed above. Alternatively, fusion proteins comprising at least the antigen binding region of an antibody disclosed herein linked to at least a functionally active portion of an enzyme as described above can be constructed using recombinant DNA techniques (see, e.g., Neuberger et al., 1984, Nature 312:604-608).

In certain embodiments, it may be desirable to use an antibody fragment, rather than an intact antibody, to increase tissue penetration, for example. It may be desirable to modify the antibody fragment in order to increase its serum half life. This can be achieved, for example, by incorporation of a salvage receptor binding epitope into the antibody fragment. In one method, the appropriate region of the antibody fragment can be altered (e.g., mutated), or the epitope can be incorporated into a peptide tag that is then fused to the antibody fragment at either end or in the middle, for example, by DNA or peptide synthesis. See, e.g., WO 96/32478.

In other embodiments, covalent modifications are also included. Covalent modifications include modification of cysteinyl residues, histidyl residues, lysinyl and amino-terminal residues, arginyl residues, tyrosyl residues, carboxyl side groups (aspartyl or glutamyl), glutaminyl and asparaginyl residues, or seryl, or threonyl residues. Another type of covalent modification involves chemically or enzymatically coupling glycosides to the antibody. Such modifications may be made by chemical synthesis or by enzymatic or chemical cleavage of the antibody, if applicable. Other types of covalent modifications of the antibody can be introduced into the molecule by reacting targeted amino acid residues of the antibody with an organic derivatizing agent that is capable of reacting with selected side chains or the amino- or carboxy-terminal residues.

Removal of any carbohydrate moieties present on the antibody can be accomplished chemically or enzymatically. Chemical deglycosylation is described by Hakimuddin et al., 1987, Arch. Biochem. Biophys. 259:52 and by Edge et al., 1981, Anal. Biochem., 118:131. Enzymatic cleavage of carbohydrate moieties on antibodies can be achieved by the use of a variety of endo- and exo-glycosidases as described by Thotakura et al., 1987, Meth. Enzymol 138:350.

Another type of useful covalent modification comprises linking the antibody to one of a variety of nonproteinaceous polymers, e.g., polyethylene glycol, polypropylene glycol, or polyoxyalkylenes, in the manner set forth in one or more of U.S. Pat. No. 4,640,835, U.S. Pat. No. 4,496,689, U.S. Pat. No. 4,301,144, U.S. Pat. No. 4,670,417, U.S. Pat. No. 4,791,192 and U.S. Pat. No. 4,179,337.

### Sequence Optimization and Amino Acid Sequence Variants

Amino acid sequence variants of an antibody can be prepared by introducing appropriate nucleotide changes into the antibody DNA, or by peptide synthesis. Such variants include, for example, deletions from, and/or insertions into and/or substitutions of, residues within the amino acid sequences of the antibodies of the examples herein.

Any combination of deletions, insertions, and substitutions is made to arrive at the final construct, provided that the final construct possesses the desired characteristics. The amino acid changes also may alter post-translational processes of the antibody, such as changing the number or position of glycosylation sites.

A useful method for identification of certain residues or regions of an antibody that are preferred locations for mutagenesis is called “alanine scanning mutagenesis,” as described by Cunningham and Wells (Science, 244:1081-1085 (1989)). Here, a residue or group of target residues are identified (e.g., charged residues such as arg, asp, his, lys, and glu) and replaced by a neutral or negatively charged amino acid (typically alanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, alanine scanning or random mutagenesis is conducted at the target codon or region and the expressed antibody variants are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody fused to an epitope tag. Other insertional variants of the antibody molecule include a fusion to the N- or C-terminus of the antibody of an enzyme or a polypeptide which increases the serum half-life of the antibody.

Another type of variant is an amino acid substitution variant. These variants have at least one amino acid residue in the antibody molecule removed and a different residue inserted in its place. The sites of greatest interest for substitutional mutagenesis include the hypervariable regions, but FR alterations are also contemplated. Conservative substitutions are shown in Table 5 under the heading of “preferred substitutions”. If such substitutions result in a change in biological activity, then more substantial changes, denominated “exemplary substitutions”, or as further described below in reference to amino acid classes, may be introduced and the products screened.

TABLE 5

| Original | Exemplary | Preferred |
|---|---|---|
| Residue | Substitutions | Substitutions |
| Ala (A) | val; leu; ile | val |
| Arg (R) | lys; gln; asn | lys |
| Asn (N) | gln; his; asp, lys; arg | gln |
| Asp (D) | glu; asn | glu |
| Cys (C) | ser; ala | ser |
| Gln (Q) | asn; glu | asn |
| Glu (E) | asp; gln | asp |
| Gly (G) | ala | ala |
| His (H) | arg; asn; gln; lys; | arg |
| Ile (I) | leu; val; met; ala; phe; norleucine | leu |
| Leu (L) | ile; norleucine; val; met; ala; phe | ile |
| Lys (K) | arg; gln; asn | arg |
| Met (M) | leu; phe; ile | leu |
| Phe (F) | tyr; leu; val; ile; ala; | tyr |
| Pro (P) | ala | ala |
| Ser (S) | thr | thr |
| Thr (T) | ser | ser |
| Trp (W) | tyr; phe | tyr |
| Tyr (Y) | phe; trp; thr; ser | phe |
| Val (V) | leu; ile; met; phe ala; norleucine; | leu |

In protein chemistry, it is generally accepted that the biological properties of the antibody can be accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;
(2) neutral hydrophilic: cys, ser, thr;
(3) acidic: asp, glu;
(4) basic: asn, gin, his, lys, arg;
(5) residues that influence chain orientation: gly, pro; and
(6) aromatic: trp, tyr, phe.

Non-conservative substitutions will entail exchanging a member of one of these classes for another class.

Any cysteine residue not involved in maintaining the proper conformation of the antibody also may be substituted, generally with serine, to improve the oxidative stability of the molecule, prevent aberrant crosslinking, or provide for established points of conjugation to a cytotoxic or cytostatic compound. Conversely, cysteine bond(s) may be added to the antibody to improve its stability (particularly where the antibody is an antibody fragment such as an Fv fragment).

A type of substitutional variant involves substituting one or more hypervariable region residues of a parent antibody. Generally, the resulting variant(s) selected for further development will have improved biological properties relative to the parent antibody from which they are generated. A convenient way for generating such substitutional variants is affinity maturation using phage display. Briefly, several hypervariable region sites (e.g., 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity). In order to identify candidate hypervariable region sites for modification, alanine scanning mutagenesis can be performed to identify hypervariable region residues contributing significantly to antigen binding. Alternatively, or in addition, it may be beneficial to analyze a crystal structure of the antigen-antibody complex to identify contact points between the antibody and the target protein. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the antibody alters the original glycosylation pattern of the antibody. By “altering” is meant deleting one or more carbohydrate moieties found in the antibody, and/or adding one or more glycosylation sites that are not present in the antibody.

In some embodiments, it may be desirable to modify the antibodies of the invention to add glycosylations sites. Glycosylation of antibodies is typically either N-linked or 0-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used. Thus, in order to glycosylate a given protein, e.g., an antibody, the amino acid sequence of the protein is engineered to contain one or more of the above-described tripeptide sequences (for N-linked glycosylation sites). The alteration may also be made by the addition of, or substitution by, one or more serine or threonine residues to the sequence of the original antibody (for O-linked glycosylation sites).

Nucleic acid molecules encoding amino acid sequence variants of the antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody.

### Polynucleotides, Vectors, Host Cells, and Recombinant Methods

Other embodiments encompass isolated polynucleotides that comprise a sequence encoding an antibody, vectors, and host cells comprising the polynucleotides, and recombinant techniques for production of the antibody. The isolated polynucleotides can encode any desired form of the antibody including, for example, full length monoclonal antibodies, Fab, Fab′, F(ab′)₂, and Fv fragments, diabodies, linear antibodies, single-chain antibody molecules, and multispecific antibodies formed from antibody fragments.

The polynucleotide(s) that comprise a sequence encoding an antibody or a fragment or chain thereof can be fused to one or more regulatory or control sequence, as known in the art, and can be contained in suitable expression vectors or host cell as known in the art. Each of the polynucleotide molecules encoding the heavy or light chain variable domains can be independently fused to a polynucleotide sequence encoding a constant domain, such as a human constant domain, enabling the production of intact antibodies. Alternatively, polynucleotides, or portions thereof, can be fused together, providing a template for production of a single chain antibody.

For recombinant production, a polynucleotide encoding the antibody is inserted into a replicable vector for cloning (amplification of the DNA) or for expression. Many suitable vectors for expressing the recombinant antibody are available. The vector components generally include, but are not limited to, one or more of the following: a signal sequence, an origin of replication, one or more marker genes, an enhancer element, a promoter, and a transcription termination sequence.

The antibodies can also be produced as fusion polypeptides, in which the antibody is fused with a heterologous polypeptide, such as a signal sequence or other polypeptide having a specific cleavage site at the amino terminus of the mature protein or polypeptide. The heterologous signal sequence selected is typically one that is recognized and processed (i.e., cleaved by a signal peptidase) by the host cell. For prokaryotic host cells that do not recognize and process the antibody signal sequence, the signal sequence can be substituted by a prokaryotic signal sequence. The signal sequence can be, for example, alkaline phosphatase, penicillinase, lipoprotein, heat-stable enterotoxin II leaders, and the like. For yeast secretion, the native signal sequence can be substituted, for example, with a leader sequence obtained from yeast invertase alpha-factor (including Saccharomyces and Kluyveromyces α-factor leaders), acid phosphatase, C. albicans glucoamylase, or the signal described in WO90/13646. In mammalian cells, mammalian signal sequences as well as viral secretory leaders, for example, the herpes simplex gD signal, can be used. The DNA for such precursor region is ligated in reading frame to DNA encoding the antibody.

Expression and cloning vectors contain a nucleic acid sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria, yeast, and viruses.

The origin of replication from the plasmid pBR322 is suitable for most Gram-negative bacteria, the 2-D. plasmid origin is suitable for yeast, and various viral origins (SV40, polyoma, adenovirus, VSV, and BPV) are useful for cloning vectors in mammalian cells. Generally, the origin of replication component is not needed for mammalian expression vectors (the SV40 origin may typically be used only because it contains the early promoter).

Expression and cloning vectors may contain a gene that encodes a selectable marker to facilitate identification of expression. Typical selectable marker genes encode proteins that confer resistance to antibiotics or other toxins, e.g., ampicillin, neomycin, methotrexate, or tetracycline, or alternatively, are complement auxotrophic deficiencies, or in other alternatives supply specific nutrients that are not present in complex media, e.g., the gene encoding D-alanine racemase for Bacilli.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid, and hygromycin. Common selectable markers for mammalian cells are those that enable the identification of cells competent to take up a nucleic acid encoding an antibody, such as DHFR (dihydrofolate reductase), thymidine kinase, metallothionein-I and -II (such as primate metallothionein genes), adenosine deaminase, ornithine decarboxylase, and the like. Cells transformed with the DHFR selection gene are first identified by culturing all of the transformants in a culture medium that contains methotrexate (Mtx), a competitive antagonist of DHFR. An appropriate host cell when wild-type DHFR is employed is the Chinese hamster ovary (CHO) cell line deficient in DHFR activity (e.g., DG44).

Alternatively, host cells (particularly wild-type hosts that contain endogenous DHFR) transformed or co-transformed with DNA sequences encoding antibody, wild-type DHFR protein, and another selectable marker such as aminoglycoside 3′-phosphotransferase (APH), can be selected by cell growth in medium containing a selection agent for the selectable marker such as an aminoglycosidic antibiotic, e.g., kanamycin, neomycin, or G418. See, e.g., U.S. Pat. No. 4,965,199.

Where the recombinant production is performed in a yeast cell as a host cell, the TRP1 gene present in the yeast plasmid YRp7 (Stinchcomb et al., 1979, Nature 282: 39) can be used as a selectable marker. The TRP1 gene provides a selection marker for a mutant strain of yeast lacking the ability to grow in tryptophan, for example, ATCC No. 44076 or PEP4-1 (Jones, 1977, Genetics 85:12). The presence of the trp1 lesion in the yeast host cell genome then provides an effective environment for detecting transformation by growth in the absence of tryptophan. Similarly, Leu2p-deficient yeast strains such as ATCC 20,622 and 38,626 are complemented by known plasmids bearing the LEU2 gene.

In addition, vectors derived from the 1.6 μm circular plasmid pKD1 can be used for transformation of Kluyveromyces yeasts. Alternatively, an expression system for large-scale production of recombinant calf chymosin was reported for K. lactis (Van den Berg, 1990, Bio/Technology 8:135). Stable multi-copy expression vectors for secretion of mature recombinant human serum albumin by industrial strains of Kluyveromyces have also been disclosed (Fleer et al., 1991, Bio/Technology 9:968-975).

Expression and cloning vectors usually contain a promoter that is recognized by the host organism and is operably linked to the nucleic acid molecule encoding an antibody or polypeptide chain thereof. Promoters suitable for use with prokaryotic hosts include phoA promoter, β-lactamase and lactose promoter systems, alkaline phosphatase, tryptophan (trp) promoter system, and hybrid promoters such as the tac promoter. Other known bacterial promoters are also suitable. Promoters for use in bacterial systems also will contain a Shine-Dalgarno (S.D.) sequence operably linked to the DNA encoding the antibody.

Many eukaryotic promoter sequences are known. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Examples of suitable promoting sequences for use with yeast hosts include the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Inducible promoters have the additional advantage of transcription controlled by growth conditions. These include yeast promoter regions for alcohol dehydrogenase 2, isocytochrome C, acid phosphatase, derivative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657. Yeast enhancers also are advantageously used with yeast promoters.

Antibody transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, or from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U.S. Pat. No. 4,419,446. A modification of this system is described in U.S. Pat. No. 4,601,978. See also Reyes et al., 1982, Nature 297:598-601, disclosing expression of human p-interferon cDNA in mouse cells under the control of a thymidine kinase promoter from herpes simplex virus. Alternatively, the Rous sarcoma virus long terminal repeat can be used as the promoter.

Another useful element that can be used in a recombinant expression vector is an enhancer sequence, which is used to increase the transcription of a DNA encoding an antibody by higher eukaryotes. Many enhancer sequences are now known from mammalian genes (e.g., globin, elastase, albumin, α-fetoprotein, and insulin). Typically, however, an enhancer from a eukaryotic cell virus is used. Examples include the SV40 enhancer on the late side of the replication origin (bp 100-270), the cytomegalovirus early promoter enhancer, the polyoma enhancer on the late side of the replication origin, and adenovirus enhancers. See also Yaniv, 1982, Nature 297:17-18 for a description of enhancing elements for activation of eukaryotic promoters. The enhancer may be spliced into the vector at a position 5′ or 3′ to the antibody-encoding sequence, but is preferably located at a site 5′ from the promoter.

Expression vectors used in eukaryotic host cells (yeast, fungi, insect, plant, animal, human, or nucleated cells from other multicellular organisms) can also contain sequences necessary for the termination of transcription and for stabilizing the mRNA.

Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding antibody. One useful transcription termination component is the bovine growth hormone polyadenylation region. See WO94/11026 and the expression vector disclosed therein. In some embodiments, antibodies can be expressed using the CHEF system. (See, e.g., U.S. Pat. No. 5,888,809; the disclosure of which is incorporated by reference herein.)

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41 P disclosed in DD 266,710 published Apr. 12, 1989), Pseudomonas such as P. aeruginosa, and Streptomyces. One preferred E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

In addition to prokaryotes, eukaryotic microbes such as filamentous fungi or yeast are suitable cloning or expression hosts for antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastors (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated antibody are derived from multicellular organisms. Examples of invertebrate cells include plant and insect cells, including, e.g., numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori (silk worm). A variety of viral strains for transfection are publicly available, e.g., the L-1 variant of Autographa californica NPV and the Bm-5 strain of Bombyx mori NPV, and such viruses may be used, particularly for transfection of Spodoptera frugiperda cells.

Plant cell cultures of cotton, corn, potato, soybean, petunia, tomato, and tobacco can also be utilized as hosts.

In another aspect, expression of antibodies is carried out in vertebrate cells. The propagation of vertebrate cells in culture (tissue culture) has become routine procedure and techniques are widely available. Examples of useful mammalian host cell lines are monkey kidney CV1 line transformed by SV40 (COS-7, ATCC CRL 1651), human embryonic kidney line (293 or 293 cells subcloned for growth in suspension culture, (Graham et al., 1977, J. Gen Virol. 36: 59), baby hamster kidney cells (BHK, ATCC CCL 10), Chinese hamster ovary cells/-DHFR1 (CHO, Urlaub et al., 1980, Proc. Natl. Acad. Sci. USA 77: 4216; e.g., DG44), mouse sertoli cells (TM4, Mather, 1980, Biol. Reprod. 23:243-251), monkey kidney cells (CV1 ATCC CCL 70), African green monkey kidney cells (VERO-76, ATCC CRL-1587), human cervical carcinoma cells (HELA, ATCC CCL 2), canine kidney cells (MDCK, ATCC CCL 34), buffalo rat liver cells (BRL 3A, ATCC CRL 1442), human lung cells (W138, ATCC CCL 75), human liver cells (Hep G2, HB 8065), mouse mammary tumor (MMT 060562, ATCC CCL51), TR1 cells (Mather et al., 1982, Annals N.Y. Acad. Sci. 383: 44-68), MRC 5 cells, FS4 cells, and human hepatoma line (Hep G2).

Host cells are transformed with the above-described expression or cloning vectors for antibody production and cultured in conventional nutrient media modified as appropriate for inducing promoters, selecting transformants, or amplifying the genes encoding the desired sequences.

The host cells used to produce an antibody described herein may be cultured in a variety of media. Commercially available media such as Ham's F10 (Sigma-Aldrich Co., St. Louis, Mo.), Minimal Essential Medium ((MEM), (Sigma-Aldrich Co.), RPMI-1640 (Sigma-Aldrich Co.), and Dulbecco's Modified Eagle's Medium ((DMEM), Sigma-Aldrich Co.) are suitable for culturing the host cells. In addition, any of the media described in one or more of Ham et al., 1979, Meth. Enz. 58: 44, Barnes et al., 1980, Anal. Biochem. 102: 255, U.S. Pat. No. 4,767,704, U.S. Pat. No. 4,657,866, U.S. Pat. No. 4,927,762, U.S. Pat. No. 4,560,655, U.S. Pat. No. 5,122,469, WO 90/103430, and WO 87/00195 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as gentamicin), trace elements (defined as inorganic compounds usually present at final concentrations in the micromolar range), and glucose or an equivalent energy source. Other supplements may also be included at appropriate concentrations that would be known to those skilled in the art. The culture conditions, such as temperature, pH, and the like, are those previously used with the host cell selected for expression, and will be apparent to the ordinarily skilled artisan.

When using recombinant techniques, the antibody can be produced intracellularly, in the periplasmic space, or directly secreted into the medium. If the antibody is produced intracellularly, the cells may be disrupted to release protein as a first step. Particulate debris, either host cells or lysed fragments, can be removed, for example, by centrifugation or ultrafiltration. Carter et al., 1992, Bio/Technology 10:163-167 describes a procedure for isolating antibodies that are secreted to the periplasmic space of E. coli.

Briefly, cell paste is thawed in the presence of sodium acetate (pH 3.5), EDTA, and phenylmethylsulfonylfluoride (PMSF) over about 30 minutes. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants. A variety of methods can be used to isolate the antibody from the host cell.

The antibody composition prepared from the cells can be purified using, for example, hydroxylapatite chromatography, gel electrophoresis, dialysis, and affinity chromatography, with affinity chromatography being a typical purification technique. The suitability of protein A as an affinity ligand depends on the species and isotype of any immunoglobulin Fc domain that is present in the antibody. Protein A can be used to purify antibodies that are based on human gamma1, gamma2, or gamma4 heavy chains (see, e.g., Lindmark et al., 1983 J. Immunol. Meth. 62:1-13). Protein G is recommended for all mouse isotypes and for human gamma3 (see, e.g., Guss et al., 1986 EMBO J. 5:1567-1575). A matrix to which an affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C_{H3} domain, the Bakerbond ABX™ resin (J. T. Baker, Phillipsburg, N.J.) is useful for purification. Other techniques for protein purification such as fractionation on an ion-exchange column, ethanol precipitation, reverse phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, typically performed at low salt concentrations (e.g., from about 0-0.25M salt).

Also included are nucleic acids that hybridize under low, moderate, and high stringency conditions, as defined herein, to all or a portion (e.g., the portion encoding the variable region) of the nucleotide sequence represented by isolated polynucleotide sequence(s) that encode an antibody or antibody fragment of the present invention. The hybridizing portion of the hybridizing nucleic acid is typically at least 15 (e.g., 20, 25, 30 or 50) nucleotides in length. The hybridizing portion of the hybridizing nucleic acid is at least 80%, e.g., at least 90%, at least 95%, or at least 98%, identical to the sequence of a portion or all of a nucleic acid encoding a polypeptide (e.g., a heavy chain or light chain variable region), or its complement. Hybridizing nucleic acids of the type described herein can be used, for example, as a cloning probe, a primer, e.g., a PCR primer, or a diagnostic probe.

The determination of percent identity or percent similarity between two sequences can be accomplished using a mathematical algorithm. A preferred, non-limiting example of a mathematical algorithm utilized for the comparison of two sequences is the algorithm of Karlin and Altschul, 1990, Proc. Natl. Acad. Sci. USA 87:2264-2268, modified as in Karlin and Altschul, 1993, Proc. Natl. Acad. Sci. USA 90:5873-5877. Such an algorithm is incorporated into the NBLAST and XBLAST programs of Altschul et al., 1990, J. Mol. Biol. 215:403-410. BLAST nucleotide searches can be performed with the NBLAST program, score=100, wordlength=12, to obtain nucleotide sequences homologous to a nucleic acid encoding a protein of interest. BLAST protein searches can be performed with the XBLAST program, score=50, wordlength=3, to obtain amino acid sequences homologous to protein of interest. To obtain gapped alignments for comparison purposes, Gapped BLAST can be utilized as described in Altschul et al., 1997, Nucleic Acids Res. 25:3389-3402. Alternatively, PSI-Blast can be used to perform an iterated search which detects distant relationships between molecules (Id.). When utilizing BLAST, Gapped BLAST, and PSI-Blast programs, the default parameters of the respective programs (e.g., XBLAST and NBLAST) can be used. Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti, 1994, Comput. Appl. Biosci. 10:3-5; and FASTA described in Pearson and Lipman, 1988, Proc. Natl. Acad. Sci. USA 85:2444-8. Within FASTA, ktup is a control option that sets the sensitivity and speed of the search. If ktup=2, similar regions in the two sequences being compared are found by looking at pairs of aligned residues; if ktup=1, single aligned amino acids are examined. ktup can be set to 2 or 1 for protein sequences, or from 1 to 6 for DNA sequences. The default if ktup is not specified is 2 for proteins and 6 for DNA. Alternatively, protein sequence alignment may be carried out using the CLUSTAL W algorithm, as described by Higgins et al., 1996, Methods Enzymol. 266:383-402.

### Non-Therapeutic Uses

The antibodies described herein are useful as affinity purification agents. In this process, the antibodies are immobilized on a solid phase such a Protein A resin, using methods well known in the art. The immobilized antibody is contacted with a sample containing to be purified, and thereafter the support is washed with a suitable solvent that will remove substantially all the material in the sample except the target protein, which is bound to the immobilized antibody. Finally, the support is washed with another suitable solvent that will release the target protein from the antibody.

It will be advantageous in some embodiments, for example, for diagnostic purposes to label the antibody with a detectable moiety. Numerous detectable labels are available, including radioisotopes, fluorescent labels, enzyme substrate labels and the like. The label may be indirectly conjugated with the antibody using various known techniques. For example, the antibody can be conjugated with biotin and any of the three broad categories of labels mentioned above can be conjugated with avidin, or vice versa.

Biotin binds selectively to avidin and thus, the label can be conjugated with the antibody in this indirect manner. Alternatively, to achieve indirect conjugation of the label with the antibody, the antibody can be conjugated with a small hapten (such as digoxin) and one of the different types of labels mentioned above is conjugated with an anti-hapten antibody (e.g., anti-digoxin antibody). Thus, indirect conjugation of the label with the antibody can be achieved.

Exemplary radioisotopes labels include ³⁵S, ¹⁴C, ¹²⁵I, ³H, and ¹³¹I. The antibody can be labeled with the radioisotope, using the techniques described in, for example, Current Protocols in Immunology, Volumes 1 and 2, 1991, Coligen et al., Ed. Wiley-Interscience, New York, N.Y., Pubs. Radioactivity can be measured, for example, by scintillation counting.

Exemplary fluorescent labels include labels derived from rare earth chelates (europium chelates) or fluorescein and its derivatives, rhodamine and its derivatives, dansyl, Lissamine, phycoerythrin, and Texas Red are available. The fluorescent labels can be conjugated to the antibody via known techniques, such as those disclosed in Current Protocols in Immunology, for example. Fluorescence can be quantified using a fluorimeter.

There are various well-characterized enzyme-substrate labels known in the art (see, e.g., U.S. Pat. No. 4,275,149 for a review). The enzyme generally catalyzes a chemical alteration of the chromogenic substrate that can be measured using various techniques. For example, alteration may be a color change in a substrate that can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light that can be measured, using a chemiluminometer, for example, or donates energy to a fluorescent acceptor.

Examples of enzymatic labels include luciferases such as firefly luciferase and bacterial luciferase (U.S. Pat. No. 4,737,456), luciferin, 2,3-dihydrophthalazinediones, malate dehydrogenase, urease, peroxidase such as horseradish peroxidase (HRPO), alkaline phosphatase, β-galactosidase, glucoamylase, lysozyme, saccharide oxidases (such as glucose oxidase, galactose oxidase, and glucose-6-phosphate dehydrogenase), heterocydic oxidases (such as uricase and xanthine oxidase), lactoperoxidase, microperoxidase, and the like. Techniques for conjugating enzymes to antibodies are described, for example, in O'Sullivan et al., 1981, Methods for the Preparation of Enzyme-Antibody Conjugates for use in Enzyme Immunoassay, in Methods in Enzym. (J. Langone & H. Van Vunakis, eds.), Academic press, N.Y., 73: 147-166.

Examples of enzyme-substrate combinations include, for example: Horseradish peroxidase (HRPO) with hydrogen peroxidase as a substrate, wherein the hydrogen peroxidase oxidizes a dye precursor such as orthophenylene diamine (OPD) or 3,3′,5,5′-tetramethyl benzidine hydrochloride (TMB); alkaline phosphatase (AP) with para-Nitrophenyl phosphate as chromogenic substrate; and β-D-galactosidase (β-D-Gal) with a chromogenic substrate such as p-nitrophenyl-β-D-galactosidase or fluorogenic substrate 4-methylumbelliferyl-β-D-galactosidase.

Numerous other enzyme-substrate combinations are available to those skilled in the art. For a general review of these, see U.S. Pat. No. 4,275,149 and U.S. Pat. No. 4,318,980.

In another embodiment, the antibody is used unlabeled and detected with a labeled antibody that binds the antibody.

The antibodies described herein may be employed in any known assay method, such as competitive binding assays, direct and indirect sandwich assays, and immunoprecipitation assays. See, e.g., Zola, Monoclonal Antibodies: A Manual of Techniques, pp. 147-158 (CRC Press, Inc. 1987).

### Diagnostic Kits

An antibody can be used in a diagnostic kit, i.e., a packaged combination of reagents in predetermined amounts with instructions for performing the diagnostic assay. Where the antibody is labeled with an enzyme, the kit may include substrates and cofactors required by the enzyme such as a substrate precursor that provides the detectable chromophore or fluorophore. In addition, other additives may be included such as stabilizers, buffers (for example a block buffer or lysis buffer), and the like. The relative amounts of the various reagents may be varied widely to provide for concentrations in solution of the reagents that substantially optimize the sensitivity of the assay. The reagents may be provided as dry powders, usually lyophilized, including excipients that on dissolution will provide a reagent solution having the appropriate concentration.

### Therapeutic Uses

In another embodiment, an antibody disclosed herein is useful in the treatment of various disorders associated with the expression of on or more target proteins. Methods for treating a disorder comprise administering a therapeutically effective amount of an antibody to a subject in need thereof.

The antibody or agent is administered by any suitable means, including parenteral, subcutaneous, intraperitoneal, intrapulmonary, intra-ocular, trans-dermal, topical, orally inhaled and intranasal, and, if desired for local immunosuppressive treatment, intralesional administration (including perfusing or otherwise contacting the graft with the antibody before transplantation). The antibody or agent can be administered, for example, as an infusion or as a bolus. Parenteral infusions include intramuscular, intravenous, intraarterial, intraperitoneal, intra-articular, or subcutaneous administration. In addition, the antibody is suitably administered by pulse infusion, particularly with declining doses of the antibody. In one aspect, the dosing is given by injections, most preferably intravenous or subcutaneous injections, depending in part on whether the administration is brief or chronic.

For the prevention or treatment of disease, the appropriate dosage of antibody will depend on a variety of factors such as the type of disease to be treated, as defined above, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments.

Depending on the type and severity of the disease, about 1 μg/kg to 20 mg/kg (e.g., 0.1-15 mg/kg) of antibody is an initial candidate dosage for administration to the patient, whether, for example, by one or more separate administrations, or by continuous infusion. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. However, other dosage regimens may be useful. The progress of this therapy is easily monitored by conventional techniques and assays.

The term “suppression” is used herein in the same context as “amelioration” and “alleviation” to mean a lessening of one or more characteristics of the disease.

The antibody composition will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the antibody to be administered will be governed by such considerations, and is the minimum amount necessary to prevent, ameliorate, or treat the disorder associated with detrimental activity.

The antibody need not be, but is optionally, formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as used hereinbefore or about from 1 to 99% of the heretofore employed dosages.

### Pharmaceutical Compositions and Administration Thereof

A composition comprising an antibody can be administered to a subject having or at risk of having an inflammatory disease, an autoimmune disease, a respiratory disease, a metabolic disorder, a disease of the central nervous system (CNS), for example a disease of the central nervous system (CNS) related to inflammation, or cancer. The invention further provides for the use of antibody in the manufacture of a medicament for prevention or treatment of an inflammatory disease, an autoimmune disease, a respiratory disease, a metabolic disorder, a disease of the central nervous system (CNS), for example a disease of the central nervous system (CNS) related to inflammation, or cancer. The term “subject” as used herein means any mammalian, e.g., humans and non-human mammals, such as primates, rodents, and dogs. Subjects specifically intended for treatment using the methods described herein include humans. The antibodies or agents can be administered either alone or in combination with other compositions in the prevention or treatment of an inflammatory disease, an autoimmune disease, a respiratory disease, a metabolic disorder, a disease of the central nervous system (CNS), for example a disease of the central nervous system (CNS) related to inflammation, or cancer. Such compositions which can be administered in combination with the antibodies or agents include methotrexate (MTX) and immunomodulators, e.g. antibodies or small molecules.

Various delivery systems are known and can be used to administer an antibody. Methods of introduction include but are not limited to intradermal, intramuscular, intraperitoneal, intravenous, subcutaneous, intranasal, intraocular, epidural, and oral routes. The antibody can be administered, for example by infusion, bolus or injection, and can be administered together with other biologically active agents such as chemotherapeutic agents. Administration can be systemic or local. In preferred embodiments, the administration is by subcutaneous injection. Formulations for such injections may be prepared in for example prefilled syringes that may be administered once every other week.

In specific embodiments, the antibody is administered by injection, by means of a catheter, by means of a suppository, or by means of an implant, the implant being of a porous, non-porous, or gelatinous material, including a membrane, such as a sialastic membrane, or a fiber. Typically, when administering the composition, materials to which the antibody or agent does not absorb are used.

In other embodiments, the antibody or agent is delivered in a controlled release system. In one embodiment, a pump may be used (see, e.g., Langer, 1990, Science 249:1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et al., 1989, J. Neurosurg. 71:105.) Other controlled release systems are discussed, for example, in Langer, supra.

An antibody can be administered as pharmaceutical compositions comprising a therapeutically effective amount of the binding agent and one or more pharmaceutically compatible ingredients.

In typical embodiments, the pharmaceutical composition is formulated in accordance with routine procedures as a pharmaceutical composition adapted for intravenous or subcutaneous administration to human beings. Typically, compositions for administration by injection are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical is administered by injection, an ampoule of sterile water for injection or saline can be provided so that the ingredients can be mixed prior to administration.

Further, the pharmaceutical composition can be provided as a pharmaceutical kit comprising (a) a container containing an antibody in lyophilized form and (b) a second container containing a pharmaceutically acceptable diluent (e.g., sterile water) for injection. The pharmaceutically acceptable diluent can be used for reconstitution or dilution of the lyophilized antibody. Optionally associated with such container(s) can be a notice in the form prescribed by a governmental agency regulating the manufacture, use or sale of pharmaceuticals or biological products, which notice reflects approval by the agency of manufacture, use or sale for human administration.

The amount of the antibody that is effective in the treatment or prevention of an immunological disorder or cancer can be determined by standard clinical techniques. In addition, in vitro assays may optionally be employed to help identify optimal dosage ranges. The precise dose to be employed in the formulation will also depend on the route of administration, and the stage of immunological disorder or cancer, and should be decided according to the judgment of the practitioner and each patient's circumstances. Effective doses may be extrapolated from dose-response curves derived from in vitro or animal model test systems.

Generally, the dosage of an antibody administered to a patient is typically about 0.1 mg/kg to about 100 mg/kg of the subject's body weight. The dosage administered to a subject is about 0.1 mg/kg to about 50 mg/kg, about 1 mg/kg to about 30 mg/kg, about 1 mg/kg to about 20 mg/kg, about 1 mg/kg to about 15 mg/kg, or about 1 mg/kg to about 10 mg/kg of the subject's body weight.

Exemplary doses include, but are not limited to, from 1 ng/kg to 100 mg/kg. In some embodiments, a dose is about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg or about 16 mg/kg. The dose can be administered, for example, daily, once per week (weekly), twice per week, thrice per week, four times per week, five times per week, six times per week, biweekly or monthly, every two months, or every three months. In specific embodiments, the dose is about 0.5 mg/kg/week, about 1 mg/kg/week, about 2 mg/kg/week, about 3 mg/kg/week, about 4 mg/kg/week, about 5 mg/kg/week, about 6 mg/kg/week, about 7 mg/kg/week, about 8 mg/kg/week, about 9 mg/kg/week, about 10 mg/kg/week, about 11 mg/kg/week, about 12 mg/kg/week, about 13 mg/kg/week, about 14 mg/kg/week, about 15 mg/kg/week or about 16 mg/kg/week. In some embodiments, the dose ranges from about 1 mg/kg/week to about 15 mg/kg/week.

In some embodiments, the pharmaceutical compositions comprising an antibody can further comprise a therapeutic agent, either conjugated or unconjugated to the binding agent. The antibody can be co-administered in combination with one or more therapeutic agents for the treatment or prevention of an inflammatory disease, an autoimmune disease, a respiratory disease, a metabolic disorder, a disease of the central nervous system (CNS), for example a disease of the central nervous system (CNS) related to inflammation, or cancer.

Such combination therapy administration can have an additive or synergistic effect on disease parameters (e.g., severity of a symptom, the number of symptoms, or frequency of relapse).

With respect to therapeutic regimens for combinatorial administration, in a specific embodiment, an antibody is administered concurrently with a therapeutic agent. In another specific embodiment, the therapeutic agent is administered prior or subsequent to administration of the antibody agent, by at least an hour and up to several months, for example at least an hour, five hours, 12 hours, a day, a week, a month, or three months, prior or subsequent to administration of the antibody.

### Articles of Manufacture

In another aspect, an article of manufacture containing materials useful for the treatment of the disorders described above is included. The article of manufacture comprises a container and a label. Suitable containers include, for example, bottles, vials, syringes, and test tubes. The containers may be formed from a variety of materials such as glass or plastic. The container holds a composition that is effective for treating the condition and may have a sterile access port. For example, the container may be an intravenous solution bag or a vial having a stopper pierceable by a hypodermic injection needle. The active agent in the composition is the antibody. The label on or associated with the container indicates that the composition is used for treating the condition of choice. The article of manufacture may further comprise a second container comprising a pharmaceutically-acceptable buffer, such as phosphate-buffered saline, Ringer's solution, and dextrose solution. It may further include other materials desirable from a commercial and user standpoint, including other buffers, diluents, filters, needles, syringes, and package inserts with instructions for use.

The invention is further described in the following examples, which are not intended to limit the scope of the invention.

### EXAMPLES

### Example 1

### Protein Expression and Purification

Nucleic acid sequences encoding variable regions were subcloned into a custom mammalian expression vectors containing constant region of IgG1 expression cassettes using standard PCR restriction enzyme based cloning techniques. The multispecific antibodies were expressed by transient transfection in Chinese hamster ovary cell line. The antibodies were initially purified by Mab Select SuRe Protein A column (GE healthcare, Piscataway, N.J.) (Brown, Bottomley et al. 1998). The column was equilibrated with Phosphate Buffer Saline (PBS), pH 7.2 and loaded with fermentation supernatant at a flow rate of 2 mL/min. After loading, the column was washed with PBS (4 CV) followed by elution in 30 mM sodium acetate, pH 3.5. Fractions containing protein peaks as monitored by Absorbance at 280 nm in Akta Explorer (GE healthcare) were pooled together and were neutralized to pH 5.0 by adding 1% of 3M sodium acetate, pH 9.0. Average recovery of the protein A purified antibody was >90%. As a polishing step, the antibodies were purified on a preparative size exclusion chromatography (SEC) using a Superdex 200 column (GE healthcare).

### Example 2

### Analytical Size Exclusion Chromatography (SEC)

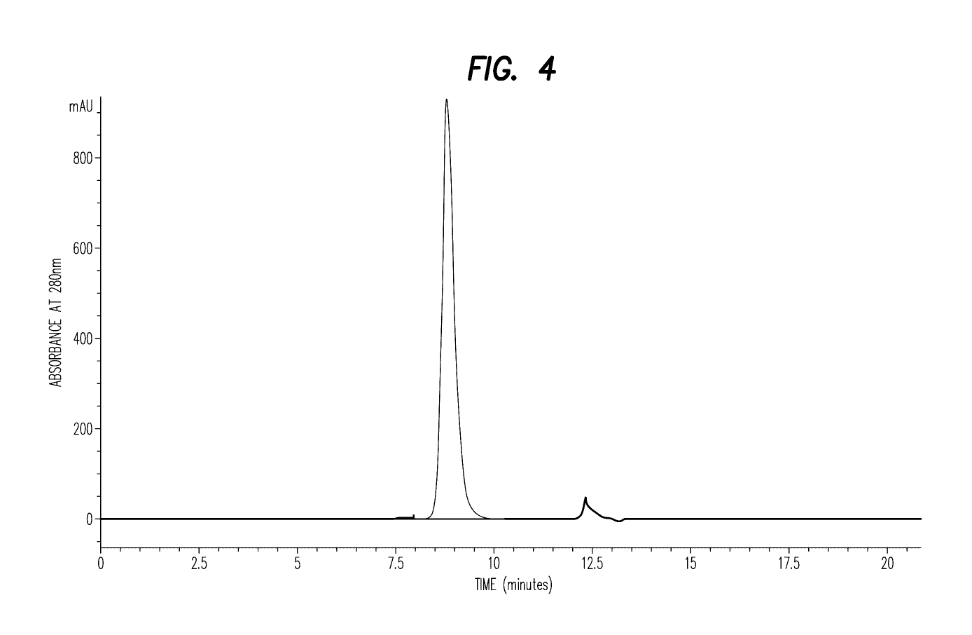

SEC-HPLC was carried out using TSKgel G3000SWXL column (7.8 mm diameter, 30 cm length, 5 μm) in 50 mM Phosphate, 200 mM Arginine, 0.05% sodium azide, pH 6.5 buffer. Flow rate was maintained at 1 mL/min and loaded sample volume was 50 μL. The elution peaks were integrated (area-under the curve) using the manufactured provided software to calculate percent monomer. The results from these experiments are shown in FIG. 4 and FIG. 8.

### Example 3

### Characterization of Homogeneity Via Analytical Ultracentrifugation (AUC)

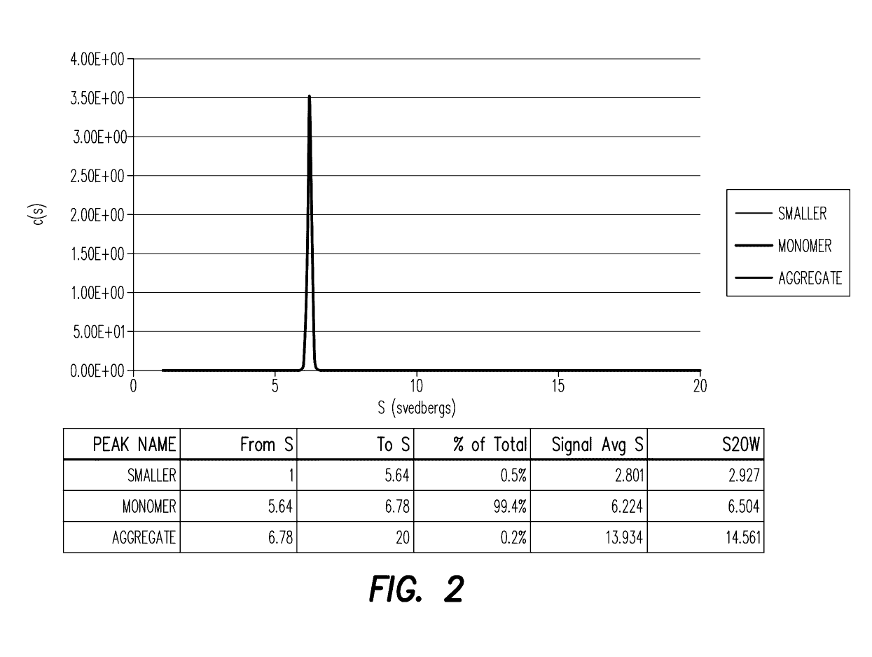

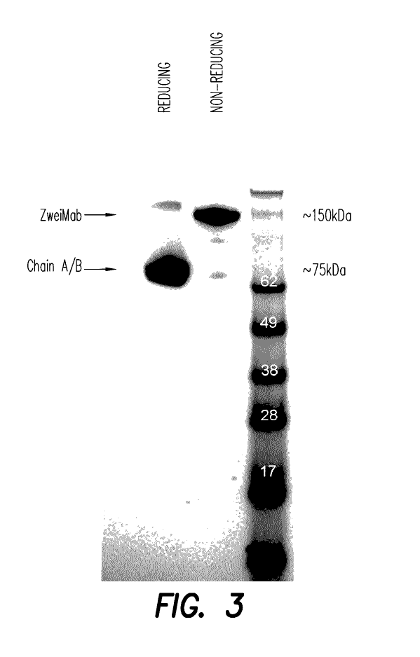

All experiments were conducted on a Beckman XLI analytical ultracentrifuge (Beckman Coulter, Inc., Fullerton, Calif.). All sedimentation velocity experiments were conducted at 40,000 rpm and 20° C. Experiments were conducted in a pH 6.0 buffer containing 20 mM Citrate and 115 mM NaCl. Data were collected at 280 nm and were analyzed using continuous c(S) model in SedFit version 12.1c. The results from these experiments are shown in FIG. 2.

### Example 4

### Differential Scanning Calorimetry (DSC)

The thermal stability of multispecific antibodies were characterized by capillary VP-DSC microcalorimeter (Microcal Inc. Northampton, Mass.). The concentration of protein was about ˜1.4 mg/mL measured at a scan rate of 1° C./min with a cell volume of 0.450 mL. Temperature scans were performed from 25 to 120° C. A buffer reference scan was subtracted from protein scan and the concentration of protein was normalized prior to thermodynamic analysis. The data was plotted in Origin 7.0 (OriginLab, Northampton, Mass.) and subsequent thermodynamic analysis was carried out on pre- and post-transition baseline corrected data. The DSC curve was fitted using non-two-state model to obtain the calorimetric enthalpy, the Van't Hoff enthalpy and apparent transition temperature (Tₘ).

Table 6 shows the assessment of thermal stability by Differential Scanning calorimetry for representative bispecific antibodies. Lowest transition temperature (CH2 domain of the Fc region) was 67.6° C. The transition temperature of the Fab and CH3-domain are ˜80° C. Incorporation of the neonatal FcRn mediated mutation (YTE) led to the destabilization of the Fc region by ˜4.6° C. The variable regions used in a bispecific antibody in this experiment are the variable regions of Certolizumab, Adalimumab, Ustekinumab or Ixekizumab, as shown in Table 3.

TABLE 6

| ZweiMab | YTE | Tₘ₁ (° C.) | Tₘ₂ (° C.) | Tₘ₃ (° C.) |
|---|---|---|---|---|
| SEQ ID | No | 67.6 | 80.3 | n/a |
| NOs: 23/24, | | | | |
| SEQ ID | | | | |
| NOs: 25/26 | | | | |
| and the linker | | | | |
| of SEQ ID: 7 in | | | | |
| both chains | | | | |
| SEQ ID | Yes | 63.0 | 67.3 | 80.3 |
| NOs: 31/24, | | | | |
| SEQ ID | | | | |
| NOs: 32/26 | | | | |
| and the linker | | | | |
| of SEQ ID: 7 in | | | | |
| both chains | | | | |
| SEQ ID | Yes | 61.8 | 65.1 | 72.7 |
| NOs: 32/26, | | | | |
| SEQ ID | | | | |
| NOs: 34/30 | | | | |
| and the linker | | | | |
| of SEQ ID: 7 in | | | | |
| both chains | | | | |

### Example 5

### Surface Plasmon Resonance (SPR)

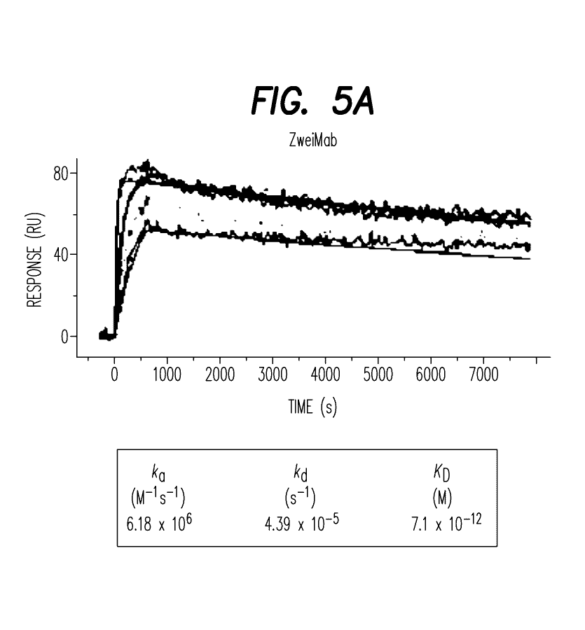

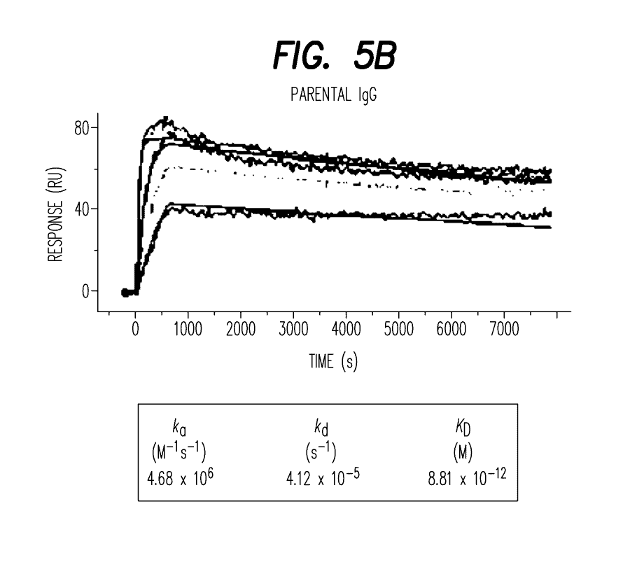

PrateOn XPR36 (Bio Rad, Hercules, Calif.) was used to measure the kinetics and affinity of target protein binding to the bispecific antibodies. Goat anti-human IgG gamma Fc specific (GAHA) (Invitrogen, Grand Island, N.Y.) was immobilized to the dextran matrix of a GLM chip (Bio Rad, Hercules, Calif.) along 6 horizontal channels using an amine coupling kit (Bio Rad, Hercules, Calif.) at a surface density between 8000 RU and 10000 RU according to the manufacturer's instructions. Bispecific antibodies were captured to the GAHA surface along 5 vertical channels at a surface density of ˜200 RU. The last vertical channel was used as a column reference to remove bulk shift. The binding kinetics of target protein with each antibody was determined by global fitting of duplicate injections of target protein at five dilutions (10, 5.0, 2.5, 1.25, 0.625, and 0 nM). The collected binding sensorgrams of target protein at five concentrations with duplicates were double referenced using inactive channel/inter-spot reference and extraction buffer reference. The referenced sensorgrams were fit into 1:1 Langmiur binding model to determine association rate (kₐ), dissociation rate (k_{d}), and dissociation constant (K_{D}). The results from these experiments are shown in FIG. 5.

### Example 6

### Serum Interference

The interaction of each of the antibodies with their (biotinylated) target protein in 1× kinetic buffer and human serum (Sigma, St. Louis, Mo.) respectively was performed on an Octet QK (ForteBio) instrument equipped with streptavidin (SA) biosensor tips (ForteBio). The sensors captured with biotinylated target protein were dipped in human serum to establish a baseline for the binding in serum before monitoring the desired interaction of antibodies with target protein. The response of the binding sensorgrams in 1× kinetic buffer and human serum at different association time points (60 sec, 120 sec, and 240 sec) were compared to determine if the antibodies bound to off-target molecules in human serum.

In Table 7, serum interference binding assay shows the lack of interference towards the target antigen binding by serum components. A representative ZweiMab bispecific antibody along with its parental IgG was assessed with binding of target antigen in PBS buffer and 90% human serum. A 1.3-fold shift in binding response was observed (concomitant to increase in refractive index of serum compared to an aqueous buffer). A similar shift was observed for the parental IgG. The variable regions used in a bispecific antibody in this experiment are the variable regions of Certolizumab and/or Adalimumab, as shown in Table 3.

TABLE 7

| | | | Binding (nm) in |
|---|---|---|---|
| | | Binding (nm) | human serum |
| Variable regions | Sensor | in PBS | (90%) |
| SEQ ID | Human | 3.04 | 3.82 |
| NOs: 23/24, SEQ ID | TNFα | | |
| NOs: 25/26 and the | | | |
| linker of SEQ ID: 7 | | | |
| in both chains | | | |
| Adalimumab | Human | 3.13 | 4.11 |
| | TNFα | | |

### Example 7

### Enzyme Linked Immunosorbent Assay (ELISA) Based Method for Protein-A Binding Assessment

An ELISA-based method was used to assess the ability of variants of multispecific antibodies to bind to protein-A. Biotinylated-protein-A was captured on Streptavidin-ELISA plate and it was incubated with 1% milk in PBST buffer to minimize non-specific binding. Subsequently, the two homodimeric variants (AA & BB) along with the heterodimeric multispecific antibody (AB) and a control IgG was incubated for 1 hour at room temperature. The plates were washed 3× with ELISA buffer and the binding was detected using an anti-kappa antibody conjugated to HRP. The results from these experiments are shown in FIG. 7b.

### Example 8

### Cynomolgus Monkey Pharmacokinetic Analysis

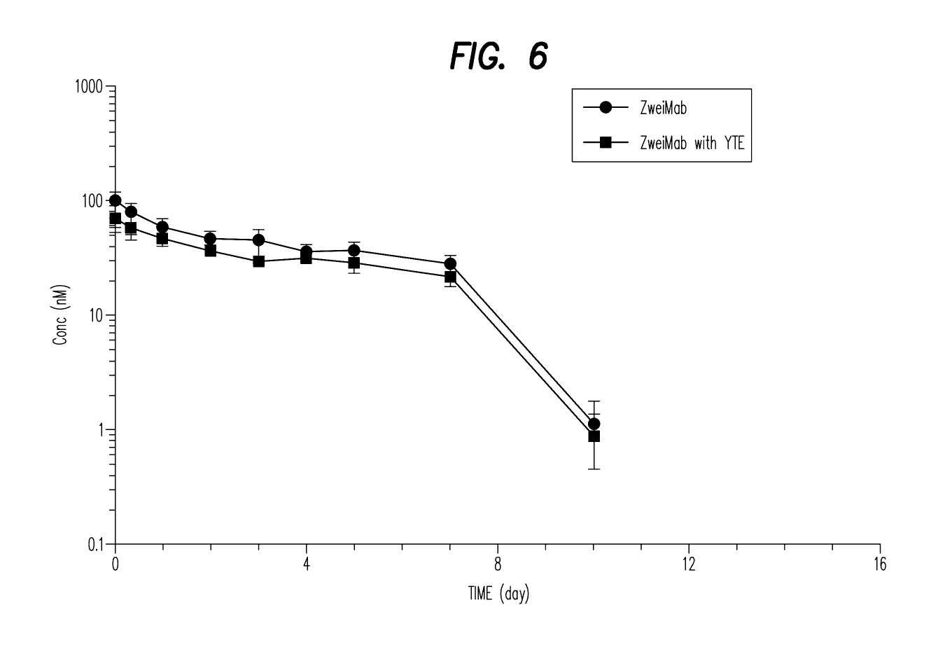

Subcutaneous pharmacokinetics of multispecific antibodies was evaluated in cynomolgus monkeys. Studies were approved by IACUC and were in compliance with USDA Animal Welfare Act (9CFR Parts 1, 2 and 3). The antibodies were administered by subcutaneous injection to the middle interscapular region. Serum concentrations of multispecific antibodies were determined using a validated, antigen-capture ELISA assay. Briefly, biotinylated target protein was immobilized on Streptavidin-coated Nunc MaxiSorp (Affimetrix eBioscience, San Diego, Calif.). The 96-well plates were washed then blocked with PBS and 2% BSA (w/v). Matrix reference standards, quality control and test samples were then diluted to a final concentration of 5% monkey serum and transferred to the blocked plate. Plates were washed prior to addition of goat anti human IgG-HRP (Southern Biotech) at a concentration of 0.05 μg/ml. Plates were washed again BioFx (SurModics, Eden Prairie, Minn.) TMBW substrate was added. Plates were allowed to develop for ˜5 minutes at room temperature prior to addition of BioFx liquid stop solution for TMB substrate (0.2M H₂SO₄) and were then read using a SpectraMax (Molecular Devices, Sunnyvale, Calif.) M5 Plate Reader at OD 450 nM. Concentrations were derived by plotting standard curve concentrations versus 450 OD signal intensity in a log-log curve fit using Softmax Pro software (Molecular Devices, Sunnyvale, Calif.). Non-compartmental pharmacokinetic analysis was performed WinNonlin (v. 5.3, Pharsight Corporation, Mountain View, Calif., USA. Areas under the serum concentration-time curve to the last quantifiable time point (AUCO-t) were calculated using the linear trapezoidal method and were extrapolated to time infinity (AUC_{inf}) using log-linear regression of the terminal portion of the individual curves to estimate the terminal half-life (t_{1/2}). The elimination rate constant (kₑₗ) was determined by least-squares regression of the log-transformed concentration data using the terminal phase, identified by inspection between days 1 and 7 and terminal half-life was equal to In2/kₑₗ. The results from these experiments are shown in FIG. 6.

Table 8 shows the pharmacokinetic profile of representative bispecific antibodies. The variable regions used in a bispecific antibody in this experiment are the variable regions of Certolizumab and Adalimumab, as shown in Table 3.

TABLE 8

| Variable | | AUCₜ | AUC_{inf} | C_{L} | V_{SS} | T_{1/2} | MRT |
|---|---|---|---|---|---|---|---|
| regions | | (nM · day) | (nM · day) | (mL/day/kg) | (day) | (day) | (day) |
| SEQ ID | Mean | 318 | 592 | 6.9 | 62.4 | 6.7 | 9.3 |
| NOs: 23/24, | SD | 53 | 109 | 1.4 | 14.3 | 2.3 | 3.2 |
| SEQ ID | | | | | | | |
| NOs: 25/26 | | | | | | | |
| and the | | | | | | | |
| linker of SEQ | | | | | | | |
| ID: 7 in both | | | | | | | |
| chains | | | | | | | |
| SEQ ID | Mean | 245 | 507 | 8.4 | 83.5 | 7.8 | 10.7 |
| NOs: 31/24, SEQ | SD | 28 | 159 | 2.8 | 6.4 | 2.7 | 3.8 |
| ID | | | | | | | |
| NOs: 32/26 | | | | | | | |
| and the | | | | | | | |
| linker of SEQ | | | | | | | |
| ID: 7 in both | | | | | | | |
| chains | | | | | | | |

### Example 9

### Immunogenicity Assessment

The bispecific antibody sequences were analyzed for potential immunogenicity using the T-regualory (Treg) adjusted scores from the EpiVax Epimatrix in silico immunogenicity prediction program.

Table 9 shows immunogenicity profile for a representative bispecific antibody.

TABLE 9
Bispecific antibody

| Mutations | Treg. Adjusted Epivax score |
|---|---|
| T366Y | −37.90 |
| Y407T | −40.61 |

Treg. Adjusted Epivax score for Fc domain (IgG1KO) −37.76

### Example 10

### Analytical Size Exclusion Chromatography (aSEC)

a) DNA Construction Methods and Cell Culture

DNA constructs were assembled using traditional cloning methods. DNA segments were either synthesized at external vendors (IDTDNA) or from PCR of previously-built in-house constructs. Segments were assembled using SOE (Splice-overlap extension) PCR and either in-fused (Clontech, cat#639638)) or ligated into restriction-enzyme digested-vectors. The standard vector used was pTT5 (National Research Council Canada) with CMV promoter, AMP gene selection marker, and OriP gene for episomal replication in CHO-E (Chinese-hamster ovary) cells. Vectors were restriction-enzyme digested with either HindIII/NheI (New England Biolabs) for IgG1KO vectors or EcoRI/ApaI (New England Biolabs) for IgG4Pro vectors. Vectors with DNA insert were transformed into Stellar cells (Clontech) and cultured overnight at 37 C with shaking. Plasmid purification minipreps (Qiagen, cat#27173) were completed and positive samples were determined by Sanger-sequencing at an external vendor (Eurofins Genomics). Cultures with sequence-verified insert were then scaled up via gigaprep plasmid purification (Qiagen, cat#12991)) or automated maxipreps (BenchPro2100 Plasmid Purification System, Thermo-Fisher). DNA was then used for transfection of CHO-E cells.

CHO-3E7 (CHO-E) cells were maintained in an actively dividing state in growth media made of FreeStyle CHO (FS-CHO; ThermoFisher Scientific) medium supplemented with 8 mM Glutamax (ThermoFisher Scientific) at 37° C., 5% CO₂, and 140 rpm shake speed. CHO-E cells were transfected at 2×10⁶ cells/mL in FS-CHO supplemented with 2 mM Glutamine (transfection culture medium). For a 35 mL CHO-E transient transfection, 35 μg of DNA containing sequences encoding the first amino acid chain and 17.5 μg DNA containing sequences encoding the second amino acid chain were diluted in 3.5 mL of OptiPro SFM in 50 mL TPP TubeSpin bioreactors. 26.25 μL of Mirus TransIT Pro transfection reagent was added to the diluted DNA mixture and the mixture gently swirled. After swirling, with incubation no longer than 1 minute, the prepared CHO-E cells were added to the DNA complexation mixture. The TubeSpin bioreactors were incubated at 37° C., 5% CO₂, 200 rpm. Four to twenty-four hours post transfection, 350 μL Gibco Anti-Clumping Agent, 350 μL Pen/Strep, and 5.25 mL CHO CD Efficient Feed B were added to the transfected cells. Twenty-four hours post-transfection the temperature was shifted to 32° C. The transfected culture was harvested after ten days or once culture is less than 60% viable. Transfections were harvested by centrifuging at 4700 rpm, 4° C. for 20 minutes. Biomass (clarified supernatant) was decanted and filtered sterilized through a 0.2 urn filter and the cell pellet was discarded. The biomass was sampled for titer by ForteBio/Pall Octet Red 96 instrument.

b) Purification & Analytical Size Exclusion Chromatography (aSEC)

30 mls of CHO-E culture supernatants were loaded onto ‘ProPlus PhyTip’ affinity columns containing 40 μl protein A resin (PhyNexus, Catalogue# PTR 91-40-07). The flow rate was 0.25 ml/min. The PhyTips were washed sequentially with 1.3 ml buffer A (DPBS), 1.3 ml buffer B (DPBS plus 1M NaCl, pH6.5) and 1.3 ml buffer A at 0.5 ml/min. Bound proteins were then eluted with 3×0.3 ml of buffer C (30 mM NaOAc, pH3.5). pH was adjusted for each eluent with 1% of buffer D (3.0 M NaOAc, pH˜9) to a final buffer of 60 mM NaOAc, pH˜5.

After measure protein concentrations, samples (˜10 μg) were run on Analytical Size Exclusion Chromatography (aSEC) columns in order to separate monomeric protein fraction from higher and lower molecular weight species. Waters BEH200 columns (4.6 mm ID×15 cm L, 1.8 um) were used on a Waters UHPLC system at a flow rate at 0.5 ml/min. The mobile phase buffer was 50 mM Sodium Phosphate pH 6.8, 200 mM Arginine, 0.05% Sodium Azide. The percent HMWs, monomers & LMWs were automatically calculated by BEH200 Processing Method.

The percentage of monomer for six proteins of the present invention is shown in Table 10. In these proteins, the first chain comprise the variable regions EpCAM, FAP or of lebrikizumab and the second chain comprise the variable regions CD33. The heavy chain constant regions are derived from IgG₁ or from IgG₄.

The pairs of amino acid chains in the proteins of the present invention tested are also indicated in Table 10.

TABLE 10

| | CD33 | |
|---|---|---|
| | (Variable regions of SEQ ID NO: 41 and 42) | |
| | IgG1 | IgG4 |
| EpCAM | 75 | 85 |
| (Variable regions of | (SEQ ID NOs: 49/51) | (SEQ ID NOs: 50/52) |
| SEQ ID NO: 41 and 42) | | |
| FAP | 56 | 73 |
| (Variable regions of | (SEQ ID NOs: 53/51) | (SEQ ID NOs: 54/51) |
| SEQ ID NO: 43 and 44) | | |
| Lebrikizumab (IL-13) | 88 | 91 |
| (Variable regions of | (SEQ ID NOs: 55/51) | (SEQ ID NOs: 56/51) |
| SEQ ID NO: 45 and 46) | | |

What is claimed is:

1. A protein comprising:
a) a first heavy chain and a first light chain forming a first binding unit specific for a first epitope, and
b) a second heavy chain and a second light chain forming a second binding unit specific for a second epitope,
wherein said first heavy chain comprises a tyrosine (Y) at position 366 [T366Y], wherein said second heavy chain comprises a threonine (T) at position 407 [Y407T], and wherein said first or said second heavy chain further comprises an arginine at position 435 [H435R] and a phenylalanine at position 436 [Y436F], or wherein said first heavy chain comprises a tryptophan (W) at position 366 [T366W], wherein said second heavy chain comprises a serine (S) at position 366 [T366S], an alanine (A) at position 368 [L368A] and a valine (V) at position 407 [Y407V], and wherein said first or said second heavy chain further comprises an arginine at position 435 [H435R] and a phenylalanine at position 436 [Y436F].

2. The protein of claim 1, wherein said second heavy chain further comprises an arginine at position 435 [H435R] and a phenylalanine at position 436 [Y436F].

3. The protein of claim 1, wherein said first and said second heavy chains further comprises YTE mutations (M252Y/S254T/T256E).

4. The protein of claim 1, wherein said first heavy chain comprises a tryptophan (W) at position 366 [T366W], wherein said second heavy chain comprises a serine (S) at position 366 [T366S], an alanine (A) at position 368 [L368A] and a valine (V) at position 407 [Y407V], wherein said second heavy chain further comprises an arginine at position 435 [H435R] and a phenylalanine at position 436 [Y436F], and wherein said heavy chains are derived from the heavy chain of an IgG₁ or IgG₄.

5. The protein of claim 1, wherein said first heavy chain comprises the amino acid sequence of SEQ ID NO: 1, 4, 36 or 37.

6. The protein of claim 1, wherein said second heavy chain comprises the amino acid sequence of SEQ ID NO: 3, 5, 38 or 39.

7. The protein of claim 1, wherein said first heavy chain comprises the amino acid sequence of SEQ ID NO: 1 or 4 and wherein said second heavy chain comprises the amino acid sequence of SEQ ID NO: 3 or 5.

8. The protein of claim 1, wherein said first heavy chain comprises the amino acid sequence of SEQ ID NO: 36 and/or wherein said second heavy chain comprises the amino acid sequence of SEQ ID NO: 38.

9. The protein of claim 1, wherein said first heavy chain comprises the amino acid sequence of SEQ ID NO: 37 and/or wherein said second heavy chain comprises the amino acid sequence of SEQ ID NO: 39.

10. The protein of claim 1, wherein said first or second light chain comprises the amino acid sequence of SEQ ID NO: 2 or 35.

11. The protein of claim 1, wherein said first heavy chain and said first light chain are covalently linked through a first linker.

12. The protein of claim 1, wherein said second heavy chain and said second light chain are covalently linked through a second linker.

13. The protein of claim 1, wherein said first heavy chain and said first light chain are covalently linked through a first linker and said second heavy chain and said second light chain are covalently linked through a second linker.

14. The protein of claim 13, wherein said first and/or said second linker comprises 26 to 42 amino acids.

15. The protein of claim 13, wherein said first and/or said second linker comprises 30 to 40 amino acids.

16. The protein of claim 13, wherein said first and/or said second linker comprises 34 to 40 amino acids.

17. The protein of claim 13, wherein said first and/or said second linker comprises 36 to 39 amino acids.

18. The protein of claim 13, wherein said first and/or said second linker comprises 38 amino acids.

19. The protein of claim 13, wherein said first and/or said second linker comprises glycine and serine amino acids.

20. The protein of claim 13, wherein said first and/or said second linker comprises the amino acid sequence of any one of SEQ ID NO:6 to SEQ ID NO:14 or SEQ ID NO:40.

21. The protein of claim 13, wherein said first and said second linker have the same length.

22. The protein of claim 13, wherein said first linker and said second linker are identical.

23. The protein of claim 13, wherein said first linker is covalently linked to the N-terminus of said first heavy chain and to the C-terminus of said first light chain and wherein said second linker is covalently linked to the N-terminus of said second heavy chain and to the C-terminus of said second light chain.

24. The protein of claim 1, wherein said first and said second epitopes are on the same target protein.

25. The protein of claim 1, wherein said first and said second epitopes are on different target proteins.

26. The protein of claim 1, further comprising a third binding unit specific to a third epitope.

27. The protein of claim 26, wherein said third binding unit is covalently linked to the C-terminus of said first or second heavy chain.

28. The protein of claim 26, wherein said third binding unit is covalently linked to the N-terminus of said first or said second light chain.

29. The protein of claim 26, further comprising a fourth binding unit specific to a fourth epitope.

30. The protein of claim 29, wherein said fourth binding unit is covalently linked to the C-terminus of said first or said second heavy chain.

31. The protein of claim 29, wherein said fourth binding unit is covalently linked to the N-terminus of said first or said second light chain.

32. The protein according to claim 29, wherein said third and/or said fourth binding unit is a scFv.

33. A pharmaceutical composition comprising a protein according to claim 1 and a pharmaceutically acceptable carrier.

34. An isolated polynucleotide comprising a sequence encoding a light chain or a heavy chain according to claim 1.

35. An expression vector comprising the polynucleotide of claim 34.

36. A host cell comprising one or more polynucleotide according to claim 34.

37. A method for producing protein comprising:
a) obtaining a host cell according to claim 36; and
b) cultivating the host cell.

38. The method according to claim 37, further comprising recovering and purifying the protein.


SEQUENCE LISTING (56 sequences)

SEQ ID NO: 1 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 107 aa, Homo sapiens [sequence omitted]

SEQ ID NO: 3 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNRFTQKSLSLSPGK

SEQ ID NO: 4 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNRFTQKSLSLSPGK

SEQ ID NO: 6 - protein, 22 aa, Artificial Sequence
  Description of Artificial Sequence Synthetic peptide
GGGGSGGGGSGGSGGSGGGGGS

SEQ ID NO: 7 - protein, 26 aa, Artificial Sequence
  Description of Artificial Sequence Synthetic peptide
GGGGSGGGGSGGGGSGGGGSGGGGGS

SEQ ID NO: 8 - protein, 30 aa, Artificial Sequence
  Description of Artificial Sequence Synthetic polypeptide
GGGGSGGGGGGSGGGGGGSGGGGSGGGGGS

SEQ ID NO: 9 - protein, 34 aa, Artificial Sequence
  Description of Artificial Sequence Synthetic polypeptide
GGGGSGGGGSGGGSGGGSGGGGSGGGGSGGGGGS

SEQ ID NO: 10 - protein, 38 aa, Artificial Sequence
  Description of Artificial Sequence Synthetic polypeptide
GGGGSGGGGSGGGSGGGSGGGSGGGGSGGGGSGGGGGS

SEQ ID NO: 11 - protein, 42 aa, Artificial Sequence
  Description of Artificial Sequence Synthetic polypeptide
GGGGSGGGGSGGGSGGGSGGGSGGGGSGGGGGSGGGSGGGGS

SEQ ID NO: 12 - protein, 34 aa, Artificial Sequence
  Description of Artificial Sequence Synthetic polypeptide
GGSEGKSSGSGSESKSTEGKSSGSGSESKSTGGS

SEQ ID NO: 13 - protein, 38 aa, Artificial Sequence
  Description of Artificial Sequence Synthetic polypeptide
GGSEGKSTSGSGSEGSKSTEGSKSSGSGSESKGSTGGS

SEQ ID NO: 14 - protein, 42 aa, Artificial Sequence
  Description of Artificial Sequence Synthetic polypeptide
GGSEGKSTSGSGSEGSKSTEGSKSEGKSTGSGSESKGSTGGS

SEQ ID NO: 15 - protein, 107 aa, Artificial Sequence [sequence omitted]

SEQ ID NO: 16 - protein, 121 aa, Artificial Sequence [sequence omitted]

SEQ ID NO: 17 - protein, 107 aa, Artificial Sequence
  Description of Artificial Sequence Synthetic polypeptide
DIQMTQSPSSLSASVGDRVTITCKASQNVGTNVAWYQQKPGKAPKALIYSASFLYSGVPYRFSGSGSGTDFTLTISSLQPEDFATYYCQQYNIYPLTFGQGTKVEIK

SEQ ID NO: 18 - protein, 118 aa, Artificial Sequence
  Description of Artificial Sequence Synthetic polypeptide
EVQLVESGGGLVQPGGSLRLSCAASGYVFTDYGMNWVRQAPGKGLEWMGWINTYIGEPIYADSVKGRFTFSLDTSKSTAYLQMNSLRAEDTAVYYCARGYRSYAMDYWGQGTLVTVSS

SEQ ID NO: 19 - protein, 107 aa, Artificial Sequence
  Description of Artificial Sequence Synthetic polypeptide
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIK

SEQ ID NO: 20 - protein, 119 aa, Artificial Sequence
  Description of Artificial Sequence Synthetic polypeptide
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSS

SEQ ID NO: 21 - protein, 112 aa, Artificial Sequence
  Description of Artificial Sequence Synthetic polypeptide
DIVMTQTPLSLSVTPGQPASISCRSSRSLVHSRGNTYLHWYLQKPGQSPQLLIYKVSNRFIGVPDRFSGSGSGTDFTLKISRVEAEDVGVYYCSQSTHLPFTFGQGTKLEIK

SEQ ID NO: 22 - protein, 119 aa, Artificial Sequence
  Description of Artificial Sequence Synthetic polypeptide
QVQLVQSGAEVKKPGSSVKVSCKASGYSFTDYHIHWVRQAPGQGLEWMGVINPMYGTTDYNQRFKGRVTITADESTSTAYMELSSLRSEDTAVYYCARYDYFTGTGVYWGQGTLVTVSS

SEQ ID NO: 23 - protein, 451 aa, Artificial Sequence
  Description of Artificial Sequence Synthetic polypeptide
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 214 aa, Artificial Sequence
  Description of Artificial Sequence Synthetic polypeptide
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 448 aa, Artificial Sequence
  Description of Artificial Sequence Synthetic polypeptide
EVQLVESGGGLVQPGGSLRLSCAASGYVFTDYGMNWVRQAPGKGLEWMGWINTYIGEPIYADSVKGRFTFSLDTSKSTAYLQMNSLRAEDTAVYYCARGYRSYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNRFTQKSLSLSPGK

SEQ ID NO: 26 - protein, 214 aa, Artificial Sequence
  Description of Artificial Sequence Synthetic polypeptide
DIQMTQSPSSLSASVGDRVTITCKASQNVGTNVAWYQQKPGKAPKALIYSASFLYSGVPYRFSGSGSGTDFTLTISSLQPEDFATYYCQQYNIYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 449 aa, Artificial Sequence
  Description of Artificial Sequence Synthetic polypeptide
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 214 aa, Artificial Sequence
  Description of Artificial Sequence Synthetic polypeptide
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 449 aa, Artificial Sequence
  Description of Artificial Sequence Synthetic polypeptide
QVQLVQSGAEVKKPGSSVKVSCKASGYSFTDYHIHWVRQAPGQGLEWMGVINPMYGTTDYNQRFKGRVTITADESTSTAYMELSSLRSEDTAVYYCARYDYFTGTGVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNRFTQKSLSLSPGK

SEQ ID NO: 30 - protein, 219 aa, Artificial Sequence
  Description of Artificial Sequence Synthetic polypeptide
DIVMTQTPLSLSVTPGQPASISCRSSRSLVHSRGNTYLHWYLQKPGQSPQLLIYKVSNRFIGVPDRFSGSGSGTDFTLKISRVEAEDVGVYYCSQSTHLPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 451 aa, Artificial Sequence
  Description of Artificial Sequence Synthetic polypeptide
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 448 aa, Artificial Sequence
  Description of Artificial Sequence Synthetic polypeptide
EVQLVESGGGLVQPGGSLRLSCAASGYVFTDYGMNWVRQAPGKGLEWMGWINTYIGEPIYADSVKGRFTFSLDTSKSTAYLQMNSLRAEDTAVYYCARGYRSYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNRFTQKSLSLSPGK

SEQ ID NO: 33 - protein, 449 aa, Artificial Sequence
  Description of Artificial Sequence Synthetic polypeptide
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 449 aa, Artificial Sequence
  Description of Artificial Sequence Synthetic polypeptide
QVQLVQSGAEVKKPGSSVKVSCKASGYSFTDYHIHWVRQAPGQGLEWMGVINPMYGTTDYNQRFKGRVTITADESTSTAYMELSSLRSEDTAVYYCARYDYFTGTGVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNRFTQKSLSLSPGK

SEQ ID NO: 35 - protein, 106 aa, Homo sapiens
GQPKAAPSVTLFPPSSEELQANKATLVCLISDFYPGAVKVAWKADGSPVNTGVETTTPSKQSNNKYAASSYLSLTPEQWKSHRSYSCQVTHEGSTVEKTVAPAECS

SEQ ID NO: 36 - protein, 329 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 37 - protein, 326 aa, Artificial Sequence
  Description of Artificial Sequence Synthetic polypeptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 38 - protein, 329 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 39 - protein, 327 aa, Artificial Sequence
  Description of Artificial Sequence Synthetic polypeptide
AASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSLG

SEQ ID NO: 40 - protein, 38 aa, Artificial Sequence
  Description of Artificial Sequence Synthetic polypeptide
GGGGSEGKSSGSGSESKSTEGKSSGSGSESKSTGGGGS

SEQ ID NO: 41 - protein, 113 aa, Mus sp.
ELVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIK

SEQ ID NO: 42 - protein, 120 aa, Mus sp.
EVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSS

SEQ ID NO: 43 - protein, 106 aa, Mus sp.
QIVLTQSPAIMSASPGEKVTMTCSASSGVNFMHWYQQKSGTSPKRWIFDTSKLASGVPARFSGSGSGTSYSLTISSMEAEDAATYYCQQWSFNPPTFGGGTKLEIK

SEQ ID NO: 44 - protein, 117 aa, Mus sp.
QVQLQQSGAELARPGASVNLSCKASGYTFTNNGINWLKQRTGQGLEWIGEIYPRSTNTLYNEKFKGKATLTADRSSNTAYMELRSLTSEDSAVYFCARTLTAPFAFWGQGTLVTVSA

SEQ ID NO: 45 - protein, 111 aa, Artificial Sequence [sequence omitted]

SEQ ID NO: 46 - protein, 118 aa, Artificial Sequence [sequence omitted]

SEQ ID NO: 47 - protein, 113 aa, Artificial Sequence
  Description of Artificial Sequence Synthetic polypeptide
DIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIK

SEQ ID NO: 48 - protein, 122 aa, Artificial Sequence
  Description of Artificial Sequence Synthetic polypeptide
QVQLVQSGAEVKKPGASVKVSCKASGYTFTNYGMNWVKQAPGQGLKWMGWINTYTGEPTYADDFKGRVTMTSDTSTSTAYLELHNLRSDDTAVYYCARWSWSDGYYVYFDYWGQGTTVTVSS

SEQ ID NO: 49 - protein, 707 aa, Artificial Sequence
  Description of Artificial Sequence Synthetic polypeptide
ELVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEGKSSGSGSESKSTEGKSSGSGSESKSTGGGGSEVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 50 - protein, 704 aa, Artificial Sequence
  Description of Artificial Sequence Synthetic polypeptide
ELVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEGKSSGSGSESKSTEGKSSGSGSESKSTGGGGSEVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 51 - protein, 709 aa, Artificial Sequence
  Description of Artificial Sequence Synthetic polypeptide
DIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEGKSSGSGSESKSTEGKSSGSGSESKSTGGGGSQVQLVQSGAEVKKPGASVKVSCKASGYTFTNYGMNWVKQAPGQGLKWMGWINTYTGEPTYADDFKGRVTMTSDTSTSTAYLELHNLRSDDTAVYYCARWSWSDGYYVYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 52 - protein, 706 aa, Artificial Sequence
  Description of Artificial Sequence Synthetic polypeptide
DIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEGKSSGSGSESKSTEGKSSGSGSESKSTGGGGSQVQLVQSGAEVKKPGASVKVSCKASGYTFTNYGMNWVKQAPGQGLKWMGWINTYTGEPTYADDFKGRVTMTSDTSTSTAYLELHNLRSDDTAVYYCARWSWSDGYYVYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSLG

SEQ ID NO: 53 - protein, 697 aa, Artificial Sequence
  Description of Artificial Sequence Synthetic polypeptide
QIVLTQSPAIMSASPGEKVTMTCSASSGVNFMHWYQQKSGTSPKRWIFDTSKLASGVPARFSGSGSGTSYSLTISSMEAEDAATYYCQQWSFNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEGKSSGSGSESKSTEGKSSGSGSESKSTGGGGSQVQLQQSGAELARPGASVNLSCKASGYTFTNNGINWLKQRTGQGLEWIGEIYPRSTNTLYNEKFKGKATLTADRSSNTAYMELRSLTSEDSAVYFCARTLTAPFAFWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 54 - protein, 694 aa, Artificial Sequence
  Description of Artificial Sequence Synthetic polypeptide
QIVLTQSPAIMSASPGEKVTMTCSASSGVNFMHWYQQKSGTSPKRWIFDTSKLASGVPARFSGSGSGTSYSLTISSMEAEDAATYYCQQWSFNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEGKSSGSGSESKSTEGKSSGSGSESKSTGGGGSQVQLQQSGAELARPGASVNLSCKASGYTFTNNGINWLKQRTGQGLEWIGEIYPRSTNTLYNEKFKGKATLTADRSSNTAYMELRSLTSEDSAVYFCARTLTAPFAFWGQGTLVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 55 - protein, 703 aa, Artificial Sequence
  Description of Artificial Sequence Synthetic polypeptide
DIVMTQSPDSLSVSLGERATINCRASKSVDSYGNSFMHWYQQKPGQPPKLLIYLASNLESGVPDRFSGSGSGTDFTLTISSLQAEDVAVYYCQQNNEDPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEGKSSGSGSESKSTEGKSSGSGSESKSTGGGGSQVTLRESGPALVKPTQTLTLTCTVSGFSLSAYSVNWIRQPPGKALEWLAMIWGDGKIVYNSALKSRLTISKDTSKNQVVLTMTNMDPVDTATYYCAGDGYYPYAMDNWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 56 - protein, 700 aa, Artificial Sequence
  Description of Artificial Sequence Synthetic polypeptide
DIVMTQSPDSLSVSLGERATINCRASKSVDSYGNSFMHWYQQKPGQPPKLLIYLASNLESGVPDRFSGSGSGTDFTLTISSLQAEDVAVYYCQQNNEDPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEGKSSGSGSESKSTEGKSSGSGSESKSTGGGGSQVTLRESGPALVKPTQTLTLTCTVSGFSLSAYSVNWIRQPPGKALEWLAMIWGDGKIVYNSALKSRLTISKDTSKNQVVLTMTNMDPVDTATYYCAGDGYYPYAMDNWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLG